SEQUENCE LISTING (11 sequences)

SEQ ID NO: 1 - DNA, 3596 bp, Homo sapiens
  CDS (26)...(3541)
caggagctcagctgcaggaggcaggatggtctggaggctggtcctgctggctctgtgggtgtggcccagcacgcaagctggtcaccaggacaaagacacgaccttcgaccttttcagtatcagcaacatcaaccgcaagaccattggcgccaagcagttccgcgggcccgaccccggcgtgccggcttaccgcttcgtgcgctttgactacatcccaccggtgaacgcagatgacctcagcaagatcaccaagatcatgcggcagaaggagggcttcttcctcacggcccagctcaagcaggacggcaagtccaggggcacgctgttggctctggagggccccggtctctcccagaggcagttcgagatcgtctccaatggccccgcggacacgctggatctcacctactggattgacggcacccggcatgtggtctccctggaggacgtcggcctggctgactcgcagtggaagaacgtcaccgtgcaggtggctggcgagacctacagcttgcacgtgggctgcgacctcatagacagcttcgctctggacgagcccttctacgagcacctgcaggcggaaaagagccggatgtacgtggccaaaggctctgccagagagagtcacttcaggggtttgcttcagaacgtccacctagtgtttgaaaactctgtggaagatattctaagcaagaagggttgccagcaaggccagggagctgagatcaacgccatcagtgagaacacagagacgctgcgcctgggtccgcatgtcaccaccgagtacgtgggccccagctcagagaggaggcccgaggtgtgcgaacgctcgtgcgaggagctgggaaacatggtccaggagctctcggggctccacgtcctcgtgaaccagcccagcgagaacctcaagagagtgtcgaatgataaccagtttctctgggagctcattggtggccctcctaagacaaggaacatgtcagcttgctggcaggatggccggttctttgcggaaaatgaaacgtgggtggtggacagctgcaccacgtgtacctgcaagaaatttaaaaccatttgccaccaaatcacctgcccgcctgcaacctgcgccagtccatcctttgtggaaggcgaatgctgcccttcctgcctccactcggtggacggtgaggagggctggtctccgtgggcagagtggacccagtgctccgtgacgtgtggctctgggacccagcagagaggccggtcctgtgacgtcaccagcaacacctgcttggggccctccatccagacacgggcttgcagtctgagcaagtgtgacacccgcatccggcaggacggcggctggagccactggtcaccttggtcttcatgctctgtgacctgtggagttggcaatatcacacgcatccgtctctgcaactccccagtgccccagatggggggcaagaattgcaaagggagtggccgggagaccaaagcctgccagggcgccccatgcccaatcgatggccgctggagcccctggtccccgtggtcggcctgcactgtcacctgtgccggtgggatccgggagcgcacccgggtctgcaacagccctgagcctcagtacggagggaaggcctgcgtgggggatgtgcaggagcgtcagatgtgcaacaagaggagctgccccgtggatggctgtttatccaacccctgcttcccgggagcccagtgcagcagcttccccgatgggtcctggtcatgcggctcctgccctgtgggcttcttgggcaatggcacccactgtgaggacctggacgagtgtgccctggtccccgacatctgcttctccaccagcaaggtgcctcgctgtgtcaacactcagcctggcttccactgcctgccctgcccgccccgatacagagggaaccagcccgtcggggtcggcctggaagcagccaagacggaaaagcaagtgtgtgagcccgaaaacccatgcaaggacaagacacacaactgccacaagcacgcggagtgcatctacctgggccacttcagcgaccccatgtacaagtgcgagtgccagacaggctacgcgggcgacgggctcatctgcggggaggactcggacctggacggctggcccaacctcaatctggtctgcgccaccaacgccacctaccactgcatcaaggataactgcccccatctgccaaattctgggcaggaagactttgacaaggacgggattggcgatgcctgtgatgatgacgatgacaatgacggtgtgaccgatgagaaggacaactgccagctcctcttcaatccccgccaggctgactatgacaaggatgaggttggggaccgctgtgacaactgcccttacgtgcacaaccctgcccagatcgacacagacaacaatggagagggtgacgcctgctccgtggacattgatggggacgatgtcttcaatgaacgagacaattgtccctacgtctacaacactgaccagagggacacggatggtgacggtgtgggggatcactgtgacaactgccccctggtgcacaaccctgaccagaccgacgtggacaatgaccttgttggggaccagtgtgacaacaacgaggacatagatgacgacggccaccagaacaaccaggacaactgcccctacatctccaacgccaaccaggctgaccatgacagagacggccagggcgacgcctgtgaccctgatgatgacaacgatggcgtccccgatgacagggacaactgccggcttgtgttcaacccagaccaggaggacttggacggtgatggacggggtgatatttgtaaagatgattttgacaatgacaacatcccagatattgatgatgtgtgtcctgaaaacaatgccatcagtgagacagacttcaggaacttccagatggtccccttggatcccaaagggaccacccaaattgatcccaactgggtcattcgccatcaaggcaaggagctggttcagacagccaactcggaccccggcatcgctgtaggttttgacgagtttgggtctgtggacttcagtggcacattctacgtaaacactgaccgggacgacgactatgccggcttcgtctttggttaccagtcaagcagccgcttctatgtggtgatgtggaagcaggtgacgcagacctactgggaggaccagcccacgcgggcctatggctactccggcgtgtccctcaaggtggtgaactccaccacggggacgggcgagcacctgaggaacgcgctgtggcacacggggaacacgccggggcaggtgcgaaccttatggcacgaccccaggaacattggctggaaggactacacggcctataggtggcacctgactcacaggcccaagaccggctacatcagagtcttagtgcatgaaggaaaacaggtcatggcagactcaggacctatctatgaccaaacctacgctggcgggcggctgggtctatttgtcttctctcaagaaatggtctatttctcagacctcaagtacgaatgcagagatatttaaacaagatttgctgcatttccggcaatgccctgtgcatgccatggtccctaga

SEQ ID NO: 2 - protein, 1172 aa, Homo sapiens
MVWRLVLLALWVWPSTQAGHQDKDTTFDLFSISNINRKTIGAKQFRGPDPGVPAYRFVRFDYIPPVNADDLSKITKIMRQKEGFFLTAQLKQDGKSRGTLLALEGPGLSQRQFEIVSNGPADTLDLTYWIDGTRHVVSLEDVGLADSQWKNVTVQVAGETYSLHVGCDLIDSFALDEPFYEHLQAEKSRMYVAKGSARESHFRGLLQNVHLVFENSVEDILSKKGCQQGQGAEINAISENTETLRLGPHVTTEYVGPSSERRPEVCERSCEELGNMVQELSGLHVLVNQPSENLKRVSNDNQFLWELIGGPPKTRNMSACWQDGRFFAENETWVVDSCTTCTCKKFKTICHQITCPPATCASPSFVEGECCPSCLHSVDGEEGWSPWAEWTQCSVTCGSGTQQRGRSCDVTSNTCLGPSIQTRACSLSKCDTRIRQDGGWSHWSPWSSCSVTCGVGNITRIRLCNSPVPQMGGKNCKGSGRETKACQGAPCPIDGRWSPWSPWSACTVTCAGGIRERTRVCNSPEPQYGGKACVGDVQERQMCNKRSCPVDGCLSNPCFPGAQCSSFPDGSWSCGSCPVGFLGNGTHCEDLDECALVPDICFSTSKVPRCVNTQPGFHCLPCPPRYRGNQPVGVGLEAAKTEKQVCEPENPCKDKTHNCHKHAECIYLGHFSDPMYKCECQTGYAGDGLICGEDSDLDGWPNLNLVCATNATYHCIKDNCPHLPNSGQEDFDKDGIGDACDDDDDNDGVTDEKDNCQLLFNPRQADYDKDEVGDRCDNCPYVHNPAQIDTDNNGEGDACSVDIDGDDVFNERDNCPYVYNTDQRDTDGDGVGDHCDNCPLVHNPDQTDVDNDLVGDQCDNNEDIDDDGHQNNQDNCPYISNANQADHDRDGQGDACDPDDDNDGVPDDRDNCRLVFNPDQEDLDGDGRGDICKDDFDNDNIPDIDDVCPENNAISETDFRNFQMVPLDPKGTTQIDPNWVIRHQGKELVQTANSDPGIAVGFDEFGSVDFSGTFYVNTDRDDDYAGFVFGYQSSSRFYVVMWKQVTQTYWEDQPTRAYGYSGVSLKVVNSTTGTGEHLRNALWHTGNTPGQVRTLWHDPRNIGWKDYTAYRWHLTHRPKTGYIRVLVHEGKQVMADSGPIYDQTYAGGRLGLFVFSQEMVYFSDLKYECRDI

SEQ ID NO: 3 - protein, 15 aa, Homo sapiens
DKDTTFDLFSISNIN

SEQ ID NO: 4 - DNA, 27 bp, Artificial Sequence
  primer for PCR
gaattcaggagctcagctgcaggaggc

SEQ ID NO: 5 - DNA, 25 bp, Artificial Sequence
  primer for PCR
gaattctagggaccatggcatgcac

SEQ ID NO: 6 - protein, 16 aa, Artificial Sequence
  Synthetically generated peptide
RESHFRGLLQNVHLVF

SEQ ID NO: 7 - protein, 18 aa, Artificial Sequence
  Synthetically generated peptide
PATCANPSFVEGECCPSC

SEQ ID NO: 8 - protein, 22 aa, Artificial Sequence
  Synthetically generated peptide
FAENETWVVDSCTTCTCKKFKT

SEQ ID NO: 9 - protein, 15 aa, Artificial Sequence
  Synthetically generated peptide
ELIGGPPKTRNMSAC

SEQ ID NO: 10 - protein, 7 aa, Artificial Sequence
  Synthetically generated peptide
WSPWAEW

SEQ ID NO: 11 - protein, 6 aa, Homo sapiens
CSVTVG

(19) United States
(12) Patent Application Publication
(10) Pub. No.: US 2005/0171016 A1
(43) Pub. Date: Aug. 4, 2005

(54) Thrombospondin-2 and uses thereof

(75) Inventors: Michael Streit, Boston, MA (US); Michael Detmar, Arlington, MA (US)

Correspondence Address: FISH & RICHARDSON PC, 225 FRANKLIN ST, BOSTON, MA, 02110, US

(73) Assignee: The General Hospital Corporation, a Massachusetts corporation

(21) Appl. No.: 10/997,699

(22) Filed: Nov. 24, 2004

Related U.S. Application Data
(63) Continuation of application No. 09/822,682, filed on Mar. 30, 2001, now abandoned.
Application No. 09/822,682 is a continuation-in-part of application No. 09/536,087, filed on Mar. 24, 2000, now abandoned.
(60) Provisional application No. 60/127,221, filed on Mar. 31, 1999.

(51) Int. Cl. A61K 38/18

(52) U.S. Cl. 514/12

(57) ABSTRACT

The invention features a method of treating a disorder characterized by unwanted angiogenesis and/or unwanted cellular proliferation, e.g., unwanted skin or prostate cell proliferation, by increasing a TSP-2 activity. The invention also features methods of identifying compounds which modulate, e.g., inhibit or promote, TSP-2 activity, and methods of evaluating if a subject is at risk for a disorder characterized by unwanted angiogenesis and/or unwanted cellular proliferation. The invention also features fragments and analogs of TSP-2 which can by used to treat such disorders.

### RELATED APPLICATIONS

This application is a continuation of U.S. patent application Ser. No. 09/822,682, filed on Mar. 30, 2001, which is a continuation-in-part of U.S. patent application Ser. No. 09/536,087, filed on Mar. 24, 2000, which claims the benefit of previously filed Provisional Application No. 60/127,221, filed Mar. 31, 1999, the entire contents of which are hereby incorporated in their entirety by reference.

### BACKGROUND OF THE INVENTION

In order to grow beyond minimal size and to metastasize, tumors need to induce the growth of new blood vessels (angiogenesis) providing a lifeline for tumor sustenance and waste disposal (Hanahan and Folkman (1996) Cell 86:353-64). Tumor development is associated with increased release of angiogenesis factors, most prominently of vascular endothelial growth factor (VEGF) (Brown et al., Exs 1997, 79:233-69). Several studies have shown that overexpression of angiogenesis factors in experimental tumors leads to enhanced tumor growth and vascularization, and therapeutic inhibition of VEGF activity has been shown to inhibit tumor growth and metastasis (Ferrara et al., (1995) Breast Cancer Res Treat 36:127-37; Claffey et al. (1996) Cancer Res 56:172-18; Skobe et al. (1997) Nat Med 3:1222-7).

Several naturally occurring angiogenesis inhibitors have been identified, including thrombospondin-1 (TSP-1) (Iruela-Arispe et al. (1991) Proc Natl Acad Sci USA, 88:5026-5030), angiostatin (O'Reilly et al. (1994) Cell 79:315-28) and endostatin (O'Reilly et al. (1997) Cell 88:277-85). TSP-1 is a 420 kd homotrimeric matricellular glycoprotein that regulates attachment, proliferation, migration and differentiation of various cell types (Bornstein et al. (1995) J Cell Biol 130:503-506). TSP-1 inhibits proliferation and migration of vascular endothelial cells in vitro and inhibits neovascularization in vivo, contributing to the normal quiescence of the vasculature (Tolsma et al. (1993) J Cell Biol 122:497-511). TSP-1 protein expression was shown to be inversely correlated to cellular differentiation in several squamous cell carcinoma (SCC) cell lines (Goodson et al., (1994) Proc Natl Acad Sci USA91:7129-7133), and was shown to induce SCC proliferation, adhesion, migration and invasion of cells in vitro (Siemeister et al. (1998) Cancer Metastasis Rev 17:241-248; Bornstein (1992) FASEB J 6:3290-3299; Gorczyca et al. (1993) Cancer Res 53:1945-51). Enhancement of in vitro tumor cell invasion by TSP-1 has also been reported for breast, lung and pancreatic carcinoma cell lines (Albo et al. (1994) Biochem Biophys Res Commun 203:857-65; Robbins et al.: Arch Pathol Lab Med 1987, 111:841-5; Albo et al.: Surgery 1997, 122:493-500; Christofori (1998) Angiogenesis 2:21-23). Based on the observation that antisense inhibition of TSP-1 in SCC resulted in suppression of tumor growth in vivo (Creamer et al. (1995) Am J Pathol 147:1661-7), it was suggested that TSP-1 may promote tumor growth (Tuszynski et al., (1996) Bioessays 18:71-6). In contrast, other studies reported that TSP-1 expression was inversely correlated with malignant progression in human lung, breast and bladder carcinoma cell lines (Zabrenetzky et al.(1994) Int J Cancer 59:191-5; Campbell et al. (1998) Cancer Res 58:1298-304).

In human skin, TSP-1 is deposited in the basement membrane (Wight et al. (1985) J Histochem Cytochem 33:295-302), contributing to the antiangiogenic barrier that separates the avascular epidermis from the vascularized dermis.

### SUMMARY OF THE INVENTION

The present invention is based, in part, on the discovery that overexpression of TSP-2 decreases tumour size in vivo. The invention features methods to modulate unwanted angiogenesis and tumour growth.

In general, the invention features, a method of treating a subject, e.g., a subject having a disorder. The disorder can be one characterized by unwanted cell proliferation or unwanted angiogenesis. The unwanted cell proliferation can be benign or malignant. The method includes increasing TSP-2 activity. Activity can be increased, e.g., by administering an agent which increases a TSP-2 activity. In a preferred embodiment, an agent which increases a TSP-2 activity can be one or more of the following: a TSP-2 polypeptide, or a biologically active fragment or analog thereof, e.g., a TSP-2 derived polypeptide or retro-inverso polypeptide thereof; a nucleic acid encoding a TSP-2 polypeptide, or a biologically active fragment or analog thereof; an agonist of TSP-2, e.g., an antibody or a small molecule having TSP-2 activity; or an agent that increases TSP-2 nucleic acid expression, e.g., a small molecule which binds to the promoter region of TSP-2.

TSP-2 activity can also be increased by controlled delivery to the subject of a TSP-2 nucleic acid, or a TSP-2 protein, fragment, or analog. A TSP-2 nucleic acid, protein, fragment, or analog can be administered to the subject in combination with a controlled release device, e.g., a biocompatible polymer, micro particle, or mesh. The device can reduce degradation and control the release of the TSP-2 nucleic acid, protein, fragment, or analog. Such a TSP-2 biocompatible controlled release system can be administered to the subject, e.g., by injection or implantation, e.g., intramuscularly, subcutaneously, intravenously, or at an organ, joint cavity, or at a lesion.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days.

The level of TSP-2 can be increased by increasing the endogenous TSP-2 activity. Activity can be increased by increasing the level of expression of the gene, e.g., by increasing transcription of the TSP-2 gene; increasing the stability of the TSP-2 mRNA, e.g., by altering the secondary or tertiary structure of the mRNA; increasing the translation of TSP-2 MRNA, e.g., by altering the sequence of the TSP-2 MRNA; and/or increasing the stability of the TSP-2 protein. Transcription of the TSP-2 gene can be increased, e.g., by altering the regulatory sequences of the endogenous TSP-2 gene. In one embodiment the regulatory sequence can be altered by: the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the TSP-2 gene to be transcribed more efficiently.

The above method can be preformed in vivo or ex vivo.

In a preferred embodiment, the agent which increases a TSP-2 activity is administered, e.g., by intravenous, intradermal, subcutaneous, oral and/or transdermal (topical) administration.

In a preferred embodiment the disorder is characterized by pre-cancerous, cancerous or neoplastic cells, or the presence of a tumour. The disorder can affect an epithelial tissue, e.g., skin, e.g., the dermis or epidermis. In other preferred embodiments, the disorder affects the breast, prostate, lung, stomach or bowel. In a preferred embodiment, the disorder is a cancerous cell growth, e.g., a squamous cell carcinoma of the skin, malignant melanoma, prostate cancer, breast cancer, colon cancer, lung cancer (e.g., non-small cell lung cancer), or Kaposi's sarcoma. In a particularly preferred example, the disorder is characterized by unwanted skin cell proliferation, e.g., cancer of the skin, e.g., a squamous cell carcinoma of the skin, or a malignant melanoma. In another preferred embodiment, the disorder is characterized by unwanted prostate cell proliferation, e.g., cancer of the prostate.

In a preferred embodiment, the method includes identifying a subject in need of increased TSP-2 activity.

In a preferred embodiment, the method includes inhibiting tumour growth or angiogenesis in a subject. The method can include identifying a subject in need of such inhibition, and increasing the level of TSP-2 activity, such that tumour growth or angiogenesis in the subject is inhibited. Inhibition of tumour growth can be measured by the size of areas of necrosis in a tumour, decrease in tumour size, and/or by decreased tumour vessel number and size.

In one embodiment, the method includes increasing TSP-2 activity, thereby inhibiting squamous cell carcinoma of the skin.

In another embodiment, the method includes increasing TSP-2 activity, thereby inhibiting prostate cancer.

In another embodiment, the method includes increasing TSP-2 activity, thereby inhibiting malignant melanoma.

In yet another embodiment, the method includes increasing TSP-2 activity, thereby inhibiting breast cancer.

In still yet another embodiment, the method includes increasing TSP-2 activity, thereby inhibiting colon cancer.

In yet another embodiment, the method includes increasing TSP-2 activity, thereby inhibiting lung cancer, e.g., non-small cell lung cancer.

In a preferred embodiment, the disorder is characterized by benign unwanted cell proliferation, e.g., unwanted skin proliferation in the skin, e.g., psoriasis or papilloma formation. The method can include increasing TSP-2 activity, thereby inhibiting unwanted proliferation, e.g., unwanted proliferation in the skin.

In one embodiment, the disorder is an inflammatory disorder associated with angiogenesis. For example, the disorder can be psoriasis, rheumatoid arthritis or multiple sclerosis. The method can include increasing TSP-2 activity, thereby treating the inflammatory disorder.

In another embodiment, the disorder is characterized by unwanted angiogenesis, e.g., unwanted angiogenesis of the eye. For example, the disorder can be a retinal disorder characterized by unwanted angiogenesis such as diabetic retinopathy. In other embodiments, the disorder can be, for example, restenosis after coronary angioplasty. The method can include increasing TSP-2 activity, thereby inhibiting angiogenesis.

In a preferred embodiment, the method further includes increasing TSP-1 activity. TSP-1 and TSP-2 activity can be increased either simultaneously or sequentially. Generally any of the methods useful for increasing TSP-2 activity described herein can be applied to TSP-1. By way of example, TSP-1 and TSP-2 activity can be increased by administering, e.g., both TSP-1 and TSP-2 polypeptides, or biologically active fragments or analogs thereof; nucleic acids that encode both TSP-1 and TSP-2 polypeptides, or biologically active fragments or analogs thereof; agonists of TSP-1 and TSP-2, e.g., antibodies or small molecules that increase the expression of TSP-1 and TSP-2; or other combinations of the elements mentioned above, e.g., a TSP-1 polypeptide and a nucleic acid which encodes TSP-2. TSP-1 activity can also be increased by controlled delivery of a TSP-1 polypeptide as described herein, or by increasing endogenous TSP-1 activity, e.g., by methods analogous to those described for TSP-2. In a preferred embodiment, the level of TSP-1 is increased over a sustained period of time, e.g., a period equal to or greater than 2, 10, 14, 30, 60, 90, or 180 days. E.g., a TSP-1 protein, fragment, or analog can be supplied, e.g., by any method described herein, over a sustained period of time, e.g., a period equal to or greater than 2, 10, 14, 30, 60, 90, or 180 days.

In a preferred embodiment, the method further includes inhibiting VEGF activity. VEGF activity can be decreased, e.g., by administering: a VEGF nucleic acid molecule, e.g., an antisense molecule or VEGF ribozyme, that can bind to cellular VEGF mRNA and inhibit expression of the protein, e.g., by inhibiting transcription of VEGF; an antibody that specifically binds to VEGF protein, e.g., an antibody that disrupts VEGF's ability to bind to its natural cellular target; a dominant negative VEGF protein or fragment thereof; or an agent which decreases VEGF nucleic acid expression, e.g., a small molecule which binds the promoter of VEGF.

In another preferred embodiment, the method includes inhibiting VEGF activity and increasing TSP-1 and/or TSP-2 activity. Inhibiting VEGF activity and increasing TSP-1 and/or TSP-2 activity can be performed by using any of the methods described herein, e.g., VEGF activity can be inhibited by using a VEGF antisense molecule, TSP-1 activity can be increased by administering a TSP-1 polypeptide and TSP-2 activity can be increased by administering a nucleic acid sequence which encodes a TSP-2 protein.

In another preferred embodiment, the method further includes administering a chemotherapeutic agent. Examples of such chemotherapeutic agents include taxol and carboplatin. The TSP-2 activity can be increased simultaneously or sequentially with administration of a chemotherapeutic agent.

In another embodiment, the method can include introducing a cell into a subject, e.g., a cell expressing TSP-2. In a preferred embodiment, the cell expresses a TSP-2 protein, or a fragment or an analog thereof. In another preferred embodiment, the cell has been genetically modified to cause the expression of TSP-2, e.g., the cell has been genetically modified to express a TSP-2 protein, or a fragment or an analog thereof, or the cell has been genetically modified to introduce a nucleic acid sequence, e.g., a regulatory sequence, e.g., a promoter or an enhancer, that causes or increases the expression of the endogenous TSP-2. In a preferred embodiment, the promoter of the endogenous TSP-2 gene has been replaced by another promoter, e.g., by a promoter from another gene. The cell can be an autologous, allogeneic, or xenogeneic cell, but is preferably autologous. The autologous cell is preferably from a subject characterized with a disorder of unwanted cell proliferation, e.g., an epithelial cell. The manipulated cell can be any cell type, e.g., a fibroblast, a keratinocyte, an epithelial cell, an endothelial cell, a glial cell, a neural cell, a lymphocyte, a bone marrow cell, and a muscle cell. Preferably the cell is an epithelial cell, e.g., an epidermal cell, a prostate epithelial cell, a mammary epithelial cell, and an intestinal epithelial cell. The cell can be introduced into a subject to increase TSP-2 activity. The cell can be a cell with unwanted proliferative characteristics or a normal cell.

In a preferred embodiment, the level of TSP-2 is increased over a sustained period of time, e.g., a period equal to or greater than 2, 10, 14, 30, 60, 90, or 180 days. E.g., a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

Although not being bound by theory, Applicants believe that the ability of TSP-2 to inhibit tumour growth occurs, at least in part, by its ability to inhibit angiogenesis.

In another aspect of the invention, the method includes treating a subject having a disorder characterized by unwanted skin cell proliferation, e.g., a cancerous skin disorder (e.g., squamous cell carcinomas of the skin or malignant melanoma), or a non-cancerous skin disorder, e.g., psoriasis or papilloma formation. The method includes increasing TSP-2 activity, e.g., by administering an agent which increases a TSP-2 activity. An agent which increases a TSP-2 activity can be one or more of: a TSP-2 polypeptide, or a biologically active fragment or analog thereof, e.g., a TSP-2 derived polypeptide or a retro-inverso polypeptide thereof; a nucleic acid encoding a TSP-2 polypeptide, or a biologically active fragment or analog thereof; an agonist of TSP-2, e.g., an antibody or a small molecule; or an agent that increases TSP-2 nucleic acid expression, e.g., a small molecule which binds to the promoter region of TSP-2. The level of TSP-2 can also be increased by controlled delivery to a subject of a TSP-2 protein, fragment, or analog, by increasing the endogenous TSP-2 activity, or by introducing a cell into a subject, e.g., a cell expressing a TSP-2 protein, or a fragment or an analog thereof, or a cell that has been genetically modified to cause the expression of TSP-2, e.g., a cell that has been genetically modified to express a TSP-2 protein, or a fragment or an analog thereof, or a cell that has been genetically modified to introduce a nucleic acid sequence, e.g., a regulatory sequence, e.g., a promoter or an enhancer, that causes or increases the expression of TSP-2 in the cell.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days. In another embodiment, a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

In another aspect of the invention, the method includes treating a subject having a disorder characterized by unwanted prostate cell proliferation, e.g., prostate cancer. The method includes increasing TSP-2 activity, e.g., by administering an agent which increases a TSP-2 activity. An agent which increases a TSP-2 activity can be one or more of: a TSP-2 polypeptide, or a biologically active fragment or analog thereof, e.g., a TSP-2 derived polypeptide or a retro-inverso polypeptide thereof; a nucleic acid encoding a TSP-2 polypeptide, or a biologically active fragment or analog thereof; an agonist of TSP-2, e.g., an antibody or a small molecule; or an agent that increases TSP-2 nucleic acid expression, e.g., a small molecule which binds to the promoter region of TSP-2. The level of TSP-2 can also be increased by controlled delivery to a subject of a TSP-2 protein, fragment, or analog, by increasing the endogenous TSP-2 activity, or by introducing a cell into a subject, e.g., a cell expressing a TSP-2 protein, or a fragment or an analog thereof, or a cell that has been genetically modified to cause the expression of TSP-2, e.g., a cell that has been genetically modified to express a TSP-2 protein, or a fragment or an analog thereof, or a cell that has been genetically modified to introduce a nucleic acid sequence, e.g., a regulatory sequence, e.g., a promoter or an enhancer, that causes or increases the expression of the endogenous TSP-2.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days. In another embodiment, a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

In another aspect, the method can be used to treat a cell characterized as having unwanted cell proliferation. The unwanted cell proliferation can be benign or malignant. The cell can be an epithelial cell, e.g., a skin or prostate cell. The method includes increasing TSP-2 activity, e.g., by administering an agent which increases a TSP-2 activity. An agent which increases a TSP-2 activity can be one or more of: a TSP-2 polypeptide, or a biologically active fragment or analog thereof, e.g., a TSP-2 derived polypeptide or a retro-inverso polypeptide thereof; a nucleic acid encoding a TSP-2 polypeptide, or a biologically active fragment or analog thereof; an agonist of TSP-2, e.g., an antibody or a small molecule; or an agent that increases TSP-2 nucleic acid expression, e.g., a small molecule which binds to the promoter region of TSP-2. TSP-2 activity can also be increased by controlled delivery to a subject of a TSP-2 protein, fragment, or analog, as described herein, or by increasing the endogenous TSP-2 activity, or by introducing a cell into a subject, e.g., a cell expressing a TSP-2 protein, or a fragment or an analog thereof, or a cell that has been genetically modified to cause the expression of TSP-2, e.g., a cell that has been genetically modified to express a TSP-2 protein, or a fragment or an analog thereof, or a cell that has been genetically modified to introduce a nucleic acid sequence, e.g., a regulatory sequence, e.g., a promoter or an enhancer, that causes or increases the expression of the endogenous TSP-2.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days. In another embodiment, a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

The method can be performed in vivo, in vitro or ex vivo.

In another aspect, the method can be used to treat an inflammatory disorder associated with angiogenesis. For example, the disorder can be psoriasis, rheumatoid arthritis or multiple sclerosis. The method includes increasing TSP-2 activity, e.g., by administering an agent which increases a TSP-2 activity. An agent which increases a TSP-2 activity can be one or more of: a TSP-2 polypeptide, or a biologically active fragment or analog thereof, e.g., a TSP-2 derived polypeptide or a retro-inverso polypeptide thereof; a nucleic acid encoding a TSP-2 polypeptide, or a biologically active fragment or analog thereof; an agonist of TSP-2, e.g., an antibody or a small molecule; or an agent that increases TSP-2 nucleic acid expression, e.g., a small molecule which binds to the promoter region of TSP-2. TSP-2 activity can also be increased by controlled delivery to a subject of a TSP-2 protein, fragment, or analog, as described herein, or by increasing the endogenous TSP-2 activity, or by introducing a cell into a subject, e.g., a cell expressing a TSP-2 protein, or a fragment or an analog thereof, or a cell that has been genetically modified to cause the expression of TSP-2, e.g., a cell that has been genetically modified to express a TSP-2 protein, or a fragment or an analog thereof, or a cell that has been genetically modified to introduce a nucleic acid sequence, e.g., a regulatory sequence, e.g., a promoter or an enhancer, that causes or increases the expression of the endogenous TSP-2.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days. In another embodiment, a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

The method can be performed in vivo, in vitro or ex vivo.

In another aspect, the method can be used to treat a disorder characterized by unwanted angiogenesis, e.g., unwanted angiogenesis of the eye. For example, the disorder can be a retinal disorder characterized by unwanted angiogenesis such as diabetic retinopathy. In other embodiments, the disorder can be, for example, restenosis after coronary angioplasty. The method includes increasing TSP-2 activity, e.g., by administering an agent which increases a TSP-2 activity. An agent which increases a TSP-2 activity can include one or more of: a TSP-2 polypeptide, or a biologically active fragment or analog thereof, e.g., a TSP-2 derived polypeptide or a retro-inverso polypeptide thereof; a nucleic acid encoding a TSP-2 polypeptide, or a biologically active fragment or analog thereof; an agonist of TSP-2, e.g., an antibody or a small molecule; or an agent that increases TSP-2 nucleic acid expression, e.g., a small molecule which binds to the promoter region of TSP-2. TSP-2 activity can also be increased by controlled delivery to a subject of a TSP-2 protein, fragment, or analog, as described herein, or by increasing the endogenous TSP-2 activity, or by introducing a cell into a subject, e.g., a cell expressing a TSP-2 protein, or a fragment or an analog thereof, or a cell that has been genetically modified to cause the expression of TSP-2, e.g., a cell that has been genetically modified to express a TSP-2 protein, or a fragment or an analog thereof, or a cell that has been genetically modified to introduce a nucleic acid sequence, e.g., a regulatory sequence, e.g., a promoter or an enhancer, that causes or increases the expression of the endogenous TSP-2.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days. In another preferred embodiment, a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

In a preferred embodiment, the agent for increasing TSP-2 activity can be applied topically. For example, the disorder is restenosis after coronary angioplasty, and the agent which increases TSP-2 activity is applied to a stent, e.g., is topically applied to a stent.

The method can be performed in vivo, in vitro or ex vivo.

In another aspect, the invention features, a method of treating a subject, e.g., a subject having an unwanted skin condition. An unwanted skin condition is a condition that affects the structure of the skin, e.g., affects the structure of the dermis or epidermis, or affects hair growth. The treatment can be administered to delay onset, decrease the likelihood of occurrence, or treat existing disorders. The condition can be caused, e.g., by a genetic factor (e.g., epidermolysis), or an environmental factor (e.g., ultraviolet (UV) radiation), or a combination of both (e.g., aging). The condition can be benign or malignant.

The method includes modulating TSP-2 activity, e.g., increasing or decreasing TSP-2 activity. In one embodiment, TSP-2 activity is increased, e.g., by administering an agent which increases a TSP-2 activity. An agent which increases a TSP-2 activity can be one or more of: a TSP-2 polypeptide, or a biologically active fragment or analog thereof, e.g., a TSP-2 derived polypeptide or retro-inverso polypeptide thereof; a nucleic acid encoding a TSP-2 polypeptide, or a biologically active fragment or analog thereof; an agonist of TSP-2, e.g., an antibody or a small molecule having TSP-2 activity; or an agent that increases TSP-2 nucleic acid expression, e.g., a small molecule which binds to the promoter region of TSP-2. The level of TSP-2 can be increased by increasing the endogenous TSP-2 activity. Activity can be increased by increasing the level of expression of the gene, e.g., by increasing transcription of the TSP-2 gene; increasing the stability of the TSP-2 MRNA, e.g., by altering the secondary or tertiary structure of the mRNA; increasing the translation of TSP-2 mRNA, e.g., by altering the sequence of the TSP-2 mRNA; or increasing the stability of the TSP-2 protein. Transcription of the TSP-2 gene can be increased, e.g., by altering the regulatory sequences of the endogenous TSP-2 gene. In one embodiment the regulatory sequence can be altered by; the addition of a positive regulatory element (such as an enhancer or a DNA-binding site for a transcriptional activator); the deletion of a negative regulatory element (such as a DNA-binding site for a transcriptional repressor) or replacement of the endogenous regulatory sequence, or elements therein, with that of another gene, thereby allowing the TSP-2 gene to be transcribed less efficiently.

In a preferred embodiment, the agent which increases a TSP-2 activity is administered, e.g., by intravenous, intradermal, subcutaneous, oral and/or transdermal (topical) administration.

In a preferred embodiment, the level of TSP-2 is increased over a sustained period of time, e.g., a period equal to or greater than 2, 10, 14, 30, 60, 90, or 180 days. E.g., a TSP-2 protein, fragment, or analog can be supplied, e.g., by any method described herein, over a sustained period of time, e.g., a period equal to or greater than 2, 10, 14, 30, 60, 90, or 180 days. In another embodiment, a cell expressing a TSP-2 protein, fragment, or analog can be supplied, e.g., by any method described herein, whereby TSP-2 is released over a sustained period of time, e.g., a period equal to or greater than 2, 10, 14, 30, 60, 90, or 180 days.

In another embodiment, TSP-2 activity is decreased, e.g., by administering an agent which decreases a TSP-2 activity. An agent which decreases a TSP-2 activity can be one or more of: a TSP-2 nucleic acid molecule, e.g., an antisense molecule or TSP-2 ribozyme, that can bind to cellular TSP-2 MRNA and inhibit expression of the protein, e.g., by inhibiting transcription of TSP-2; an antibody that specifically binds to a TSP-2 protein, e.g., an antibody that disrupts TSP's ability to bind to its natural cellular target; a dominant negative TSP-2 protein or fragment thereof; or an agent which decreases TSP-2 nucleic acid expression, e.g., a small molecule which binds the promoter of TSP-2. The level of TSP-2 can be decreased by decreasing the endogenous TSP-2 activity. Activity can be decreased by decreasing the level of expression of the gene, e.g., by decreasing transcription of the TSP-2 gene; decreasing the stability of the TSP-2 mRNA, e.g., by altering the secondary or tertiary structure of the mRNA; decreasing the translation of TSP-2 MRNA, e.g., by altering the sequence of the TSP-2 mRNA; or decreasing the stability of the TSP-2 protein. Transcription of the TSP-2 gene can be decreased, e.g., by altering the regulatory sequences of the endogenous TSP-2 gene. In one embodiment the regulatory sequence can be altered by; the addition of a negative regulatory sequence (such as a DNA-binding site for a transcriptional repressor).

In a preferred embodiment, the agent which decreases a TSP-2 activity is administered, e.g., by intravenous, intradermal, subcutaneous, oral and/or transdermal (topical) administration.

The above method can be performed in vivo or ex vivo.

In a preferred embodiment the method includes identifying a subject in need of modulation of TSP-2 activity, e.g., increasing TSP-2 activity.

In a preferred embodiment, the method includes treating a subject with abnormal or undesirable skin structure. Abnormal or undesirable skin structure can be caused, e.g., by genetic or environmental factors, e.g., aging or UV damage. The method can include identifying a subject in need of such treatment, and increasing the level of TSP-2 activity, such that the abnormal or undesirable skin structure is treated.

In one embodiment, the method includes increasing TSP-2 activity, thereby treating aged skin.

In one embodiment, the method includes increasing TSP-2 activity, thereby treating psoriasis.

In one embodiment, the method includes increasing TSP-2 activity, thereby treating rosecea dermatosis.

In another embodiment, the method includes increasing TSP-2 activity, thereby treating skin damage caused by photoradiation, e.g., UV radiation.

In another embodiment, the method includes increasing TSP-2 activity, thereby treating abnormal hair growth.

In a preferred embodiment, the method further includes increasing TSP-1 activity. TSP-1 and TSP-2 activity can be increased either simultaneously or sequentially. Generally any of the methods useful for increasing TSP-2 activity can be applied to TSP-1. By way of example, TSP-1 and TSP-2 activity can be increased by administering, e.g., both TSP-1 and TSP-2 polypeptides, or biologically active fragments or analogs thereof; nucleic acids that encode both TSP-1 and TSP-2 polypeptides, or biologically active fragments or analogs thereof; agonists of TSP-1 and TSP-2, e.g., antibodies or small molecules that increase the expression of TSP-1 and TSP-2; or other combinations of the elements mentioned above, e.g., a TSP-1 polypeptide and a nucleic acid which encodes TSP-2. TSP-1 activity can also be increased by use of biocompatible systems for controlled release of TSP-1, or by increasing endogenous TSP-1 activity, e.g., by methods analogous to those described for TSP-2.

In another embodiment, the method can include introducing a cell into a subject, e.g., a cell that expresses TSP-2. In a preferred embodiment, the cell has been genetically modified to cause the expression of a TSP-2 protein, fragment or an analog thereof. For example, the cell has been genetically modified to express a TSP-2 protein, or a fragment or an analog thereof, or the cell has been genetically modified to introduce a nucleic acid sequence, e.g., a regulatory sequence, e.g., a promoter or an enhancer, that causes or increases the expression of the endogenous TSP-2.

The cell can be an autologous, allogeneic, or xenogeneic cell, but is preferably autologous. The autologous cell is preferably from a subject characterized with an unwanted skin condition, e.g., subject with psoriasis.

In another aspect, the invention features, a method of evaluating a cell, a tissue, or a subject for the presence of TSP-2 RNA; TSP-2 DNA; or TSP-2 protein. In a preferred embodiment, the subject has been diagnosed to be at risk for unwanted skin or prostate cell proliferation, e.g., squamous cell carcinoma, melanoma, or prostate cancer. In a preferred embodiment, the tissue at risk is epithelial, e.g., skin, or prostate tissue. In a preferred embodiment, the method includes contacting a biological sample (e.g., a cell sample) with a compound or an agent capable of detecting TSP-2 protein or TSP-2 nucleic acid, e.g., mRNA, such that the presence of TSP-2 nucleic acid or protein is detected in the biological sample. The compound or agent can be, for example, a nucleic acid probe, e.g., a labeled nucleic acid probe, capable of hybridizing to TSP-2 mRNA or an antibody, e.g., a labeled antibody, capable of binding to TSP-2 protein.

In one embodiment, the method can be used to evaluate if a subject is at risk for unwanted proliferation, e.g., at risk for developing a tumour. A decrease in TSP-2 activity is indicative of a subject that is at risk. In another embodiment, the method can be used for characterizing or staging a disorder or disease state, e.g., staging a tumor, e.g., a carcinoma, e.g., by determining whether the tumor is at an early stage or an advanced stage, e.g., metastatic stage. A relatively lower level of a TSP-2 indicates a relatively advanced disease state.

In one embodiment, the method further includes evaluating a control, e.g., the control can be a non-cancerous cell or tissue.

In another embodiment, the method can be used to evaluate a cell, e.g., an epithelial cell, e.g., a skin cell or a prostate cell e.g., to determine if the cell, is cancerous. The method includes providing a cell from a tissue, e.g., an epithelial tissue, which is suspected of being cancerous, contacting the MRNA of said cell with a single-stranded nucleic acid probe which can hybridize under stringent conditions to a TSP-2 nucleic acid sequence and comparing the amount of hybridization of said probe to the mRNA of the cell with the amount of hybridization of said probe to the mRNA of a control cell, e.g., a normal cell. A less amount of hybridization (e.g., as determined by signal intensity) in the cell as compared to the control cell is an indication that the test cell is cancerous. The probe can be of any length, e.g., the probe can be 20, 30, 50, 60 or more nucleotides in length. The hybridization can be performed in situ or can be performed as a Northern analysis.

In another embodiment, the method can be used to evaluate a cell, e.g., an epithelial cell, e.g., a skin cell or a prostate cell e.g., to determine if the cell, is cancerous. The method includes providing a cell, e.g., an epithelial cell, e.g., a skin cell or a prostate cell, said cell suspected of being cancerous, contacting proteins of the cell with an antibody which forms an immunocomplex with TSP-2, comparing the amount of immunocomplex formation in the test cell with the amount of immunocomplex formation in a control cell, e.g., a normal cell. A lower amount of immunocomplex formation in the cell of interest, as compared to the control cell, is an indication that the cell of interest is cancerous. Kits for detecting TSP-2 nucleic acid or protein in a biological sample are also within the scope of the invention. The kit can include a probe that can selectively bind a TSP-2 nucleic acid sequence or protein. The probe can be a labeled probe, e.g., a labeled antibody. The kit may also include standards and controls, e.g., a kit can include a wild-type TSP-2 nucleic acid sequence. The kit can also include an instruction leaflet that outlines how to use the components of the kit for detecting TSP-2.

In another aspect, the invention features, a method of evaluating a candidate compound. The method is useful for identifying a compound, e.g., a TSP-2 polypeptide, or a fragment or analog thereof, which can be used to treat a disorder characterized by unwanted proliferation, e.g., an epithelial cell disorder, e.g., a skin or a prostate disorder. The method can evaluate the ability of the compound to increase TSP-2 activity, e.g., by increasing the expression of the TSP-2 gene or the activity of the TSP-2 protein. The method includes: providing a cell, a tissue, or a subject, treating the cell or the tissue, or the subject with a candidate compound; and determining the level of TSP-2 RNA, TSP-2 DNA or TSP-2 protein. The method can further include evaluating a control cell, tissue or subject, e.g., an identical cell which, e.g., is not treated with the candidate compound. An increase in the amount of TSP-2 activity in the cell tissue or subject treated with the compound in comparison to the control is indicative of a useful compound, e.g., for the treatment of unwanted cell proliferation, e.g., a benign or malignant unwanted cell proliferation. The method can further include testing the compound for the ability of the compound to inhibit angiogenesis or tumour growth, e.g., a skin or a prostate tumour. The mouse tumour model described herein is useful for this purpose. In a preferred embodiment the compound is a fragment or an analog of TSP-2.

The invention also features methods for identifying a compound which interacts with a TSP-2 protein. In a preferred embodiment, the method can include the steps of contacting the TSP-2 protein with the compound under conditions which allow binding of the compound to the TSP-2 protein to form a complex, and detecting the formation of a complex of the TSP-2 protein and the compound in which the ability of the compound to bind to the TSP-2 protein is indicated by the presence of the compound in the complex. Methods for identifying a compound or agent can be performed, for example, using a cell free assay. For example, TSP-2 can be immobilized to a suitable substrate, e.g., glutathione sepharose beads or glutathione derivatized microtitre plates, using a fusion protein which allows for TSP-2 to bind to the substrate, e.g., a glutathoine-S-transferase/TSP-2 fusion protein. The mouse tumour model described herein is useful for evaluating if the compound identified can inhibit unwanted cell proliferation, e.g., a benign or malignant unwanted cell proliferation, e.g., tumour growth. In a preferred embodiment the compound is a fragment or an analog of TSP-2.

In another embodiment, a compound which interacts with a TSP-2 protein can be identified using a cell-based assay. These methods can include identifying a compound based on its ability to promote, a biological activity of TSP-2. In a preferred embodiment, the compound modulates the biological activities of TSP-2. In a preferred embodiment, the compound is a fragment or an analog of TSP-2.

In another aspect, the invention features, a method for identifying compounds which increase TSP-2 nucleic acid expression. In a preferred embodiment, nucleic acid expression can be evaluated using a nucleic acid probe, e.g., a labeled probe, capable of hybridizing to a TSP-2 nucleic acid molecule, e.g., TSP-2 niRNA. In another preferred embodiment, TSP-2 nucleic acid expression, e.g., DNA expression, can be evaluated by contacting a compound with a TSP-2 nucleic acid molecule, e.g., a regulatory sequence of a TSP-2 nucleic acid molecule, and evaluating TSP-2 transcription, in vitro or in vivo. TSP-2 transcription can be evaluated, for example, by detecting the production of TSP-2 protein, e.g., using an antibody, e.g., a labeled antibody, or by determining a cell activity, e.g., using a marker gene, e.g., a lacZ gene, fused to the regulatory sequence of TSP-2 and following production of the marker. The method can further include testing the compound for the ability of the compound to inhibit tumour growth, e.g., a skin or a prostate tumour. The mouse tumour model described herein is useful for evaluating if the compound identified can inhibit unwanted cell proliferation, e.g., a benign or malignant unwanted cell proliferation, e.g., tumour growth. In a preferred embodiment, the compound is a fragment or an analog of TSP-2.

Another aspect of the invention features a method for in vivo evaluation of a candidate compound. In one embodiment, the method can be used to evaluate if a candidate compound increases TSP-2 activity and thereby inhibits unwanted cell proliferation. The unwanted cell proliferation can be benign or malignant. The method can include the steps of; introducing a cell characterized by unwanted cell proliferation (e.g., unwanted epithelial cell proliferation, e.g., unwanted skin or prostate cell proliferation, e.g., a carcinoma such as a squamous cell carcinoma, e.g., a A431 cell line, or a melanoma, e.g., a MeWo cell line) into an animal (e.g., an immunodeficient animal, e.g., a mouse such as a nude mouse), the TSP-2 activity of that cell being down-regulated compared to a normal cell of the same type of tissue; and allowing unwanted cell proliferation, e.g., a carcinoma formation; treating the cells with a candidate compound and determining TSP-2 activity. The method can further include; determining whether the compound affects the rate of proliferation or metastasis of the carcinoma cell in the animal, e.g., by the identification of areas of necrosis in the tumour or by the determination of tumour size. A decrease in the rate of proliferation or metastasis in the presence of the compound is an indication that the compound can be used to treat carcinomas. In another embodiment, the method can be used to determine if a candidate compound which has the ability to increase TSP-2 activity in one particular form of unwanted cell proliferation (e.g., a skin carcinoma) can be used to treat a carcinoma of another cell type (e.g., a prostate carcinoma).

The invention also features a method for evaluating a subject at risk for a disorder characterized by aberrant or abnormal TSP-2 nucleic acid expression and/or TSP-2 protein activity, e.g., a disorder associated with abnormal cell proliferation (e.g., cancer, e.g., cancer of the skin or prostate). The method includes evaluating, e.g., detecting, a genetic lesion in the TSP-2 gene, or evaluating, e.g., detecting, misexpression of the TSP-2 gene, thereby determining if a subject is at risk for (e.g., has or is predisposed to have) the disorder. In a preferred embodiment, the method includes evaluating, e.g., in a sample of cells from the subject, the presence or absence of a genetic lesion, e.g., a lesion characterized by an alteration affecting the gene encoding a TSP-2 protein, or evaluating the misexpression of the TSP-2 gene. Genetic lesions can be evaluated, e.g., by contacting the sample with a nucleic acid probe capable of hybridizing to TSP-2 mRNA, e.g., a labeled probe. Expression can be evaluated with an antibody capable of binding to TSP-2 protein, e.g., a labeled antibody. In a preferred embodiment, the method can also be used in fetal or neonatal diagnosis.

In another aspect, the invention features a composition, e.g., a therapeutic composition, for inhibiting unwanted proliferation comprising: TSP-2 or a therapeutically active fragment or analog thereof, e.g., a TSP-2 derived peptide or retro-inverso peptide thereof; a nucleic acid that encodes TSP-2 or a therapeutically active fragment or analog thereof; a compound that increases the level of expression of a TSP-2 gene or activity; and one or more additional components (e.g., a carrier, diluent or solvent). The additional component can be one which renders the composition useful for in vitro and in vivo pharmaceutical or veterinary use.

In another aspect, the invention features an isolated nucleic acid molecule which comprises the coding region of TSP-2, or a sequence which encodes a fragment or a peptide-based analog of TSP-2. The nucleic acid can include a 5′ or 3′ nucleic acid sequence not present in the native TSP-2 human sequence. In one embodiment, the nucleic acid encoding human TSP-2 includes a functional regulatory sequence, e.g., a 5′ and/or a 3′ sequence which modulates expression of TSP-2. In one embodiment the control sequence can be an endogenous regulatory sequence. In another embodiment the regulatory sequence can be a heterologous regulatory sequence. The heterologous regulatory sequence can be a human or non-human regulatory sequence, or a combination of both. A regulatory sequence can include one or more elements of a regulatory sequence, e.g., the regulatory sequence can include a promoter, an enhancer, an insulator, or a DNA binding element.

In one embodiment, the nucleic acid molecule has more than 239 bp, 250 bp or 300 bp of the 5′ native TSP-2 regulatory sequence.

In another embodiment, the nucleic acid molecule has more than 2036 bp, 2500 bp or 3000 bp of the 3′ native TSP-2 regulatory sequence.

In another embodiment, the nucleic acid molecule contains a secretion signal sequence. The secretion signal sequence can be the native secretion signal of the TSP-2 human gene or can be a heterologous signal sequence. In a preferred embodiment, the secretion signal is chosen to be functional in the cell type in which TSP-2 is expressed.

In another preferred embodiment, the sequence which encodes the coding region of TSP-2, or a sequence which encodes a fragment or an analog of the coding region of TSP-2: hybridizes, preferably under stringent conditions to SEQ ID NO:2; has at least 60, 65%, 70%, 75% 80%, 85%, 90%, 95%, 96%, 97%, 98% or 99% sequence identity to the nucleotide sequence shown in SEQ ID NO:1. In other preferred embodiments, the isolated nucleic acid molecule encodes the amino acid sequence of SEQ ID NO:2.

In one embodiment, the nucleic acid sequence encodes a protein that has at least 60%, 70%, 80%, 85%, 90%, 95%, 96%, 97%, 98% or 99% sequence identity to the amino acid sequence of SEQ ID NO:2. In another preferred embodiment, the nucleic acid that encodes the coding region of TSP-2, encodes a full-length protein that is substantially homologous to the entire amino acid sequence of SEQ ID NO:2. In another embodiment, the nucleic acid encodes a mammalian protein, which is substantially homologous to the amino acid sequence of SEQ ID NO:2, or a portion thereof.

In a preferred embodiment, the encoded TSP-2 protein or encoded fragment or analog of TSP-2 differs in amino acid sequence at least by 1 to as many as (but not more than) 2, 3, 5, 10, 20 or 40 residues from a sequence in SEQ ID NO:2. In a preferred embodiment, the differences, however, are such that: the TSP-2 encoded protein exhibits a TSP-2 biological activity, e.g., the encoded TSP-2 protein or a fragment or an analog thereof retains a biological activity of a naturally occurring TSP-2, e.g., the TSP-2 protein, or a fragment or an analog thereof, can reduce the growth and size of a tumour. A difference can be a substitution, addition or deletion of an amino acid. If the difference is a substitution, the substitutution can be a conservative change.

In a preferred embodiment, the nucleic acid that encodes the coding region of TSP-2 protein, or encodes a fragment or analog thereof, differs in its nucleic acid sequence by at least 1 to as many as (but not more than) 2, 3, 9, 15, 20, 50 or 120 nucleotides from a sequence in SEQ ID NO: 1. In a preferred embodiment, the differences, however, are such that: the nucleic acid encoding TSP-2, or a fragment thereof, encodes a TSP-2 that exhibits a TSP-2 biological activity, e.g., the encoded TSP-2 protein, or a fragment or an analog thereof retains a biological activity of a naturally occurring TSP-2, or fragment thereof, e.g., the TSP-2 protein can reduce the growth and size of a tumour. A difference can be a substitution, addition or deletion of a nucleic acid sequence.

In preferred embodiments, the encoded polypeptide includes all or a fragment of an amino acid sequence from SEQ ID NO:2, fused, e.g., in reading frame, to additional amino acid residues, preferably to residues encoded by genomic DNA 5′ to the genomic DNA which encodes a sequence from SEQ ID NO:2.

In a preferred embodiment, the TSP-2 polypeptide includes a domain that includes at least one, two or three type 1 repeat(s). Preferably, a type 1 repeat is about 40 to 60, 45 to 55, 47 to 52 amino acids in length, and preferably has about 70%, 80%, 90% or 95% sequence identity with a type 1 repeat of SEQ ID NO:2. For example, a type 1 repeat can be found at about amino acids 382 to 429 of SEQ ID NO:2; about amino acids 438 to 490 of SEQ ID NO:2; about amino acids 495 to 547 of SEQ ID NO:2. A type 1 repeat of TSP-2 may have one or more of the following activities: (i) may bind the membrane protein CD36; (ii) may promote an inhibitory effect of TSP-2 on endothelial cell migration; (iii) may induce cell apoptosis, e.g., endothelial cell apoptosis; (iv) may have anti-angiogenic activity of TSP-2; or (v) may inhibit unwanted cell proliferation, e.g., a benign or malignant unwanted cell proliferation, e.g., tumour growth. In a preferred embodiment, a TSP-2 peptide is about 4, 5, 6, 7, 8, 10, 15, 20 or 50 amino acids in length and contains a sequence which inhibits endothelial cell migration. For example, the peptide can include a PWAEW sequence (about amino acid residues 386 to 390 of SEQ ID NO:2), or the fragment can include a WSPWAEW sequence (about amino acids 384 to 390 of SEQ ID NO:2), or conservative substitutions of either sequence. Other peptides can include 4, 5 or 6 amino acids from a WSPWAEW sequence or conservative substitutions thereof. In another embodiment, a TSP-2 peptide includes about 5 to 50 amino acids of the type 1 repeat of TSP-2, or about 5 to 50 amino acids of TSP-2 sequence on one or both sides of the type 1 repeat. In a preferred embodiment, the fragment is 4, 5, 6, 7, 10, 15, 20 or 50 amino acids in length and contains a sequence which contains a receptor binding sequence, e.g., a CSVTVG sequence, which binds CD36.

The invention also features fragments and analogs of TSP-2 polypeptides, preferably having at least one biological activity of a TSP-2 polypeptide. In one embodiment, a fragment or an anolog of TSP-2 has an amino acid sequence that is at least 60%, 80%, 90%, 95%, 98%, or 99% homologous to an amino acid sequence of SEQ ID NO:2; or an amino acid sequence essentially the same as an amino acid sequence in SEQ ID NO:2. A fragment or an analog of TSP-2 can be a polypeptide of at least 5, 10, 20, 50, 100, 150, 170, 200, or 250 amino acids in length; at least 5, preferably at least 10, more preferably at least 20, most preferably at least 50, 100, 150, 200, 210 or 250 contiguous amino acids from SEQ ID NO:2. In a preferred embodiment; a fragment or analog is at least 4, 5, 10, 15, 20, 25 amino acids in length, but no more than 100 amino acids in length; and has the ability to act as an agonist of a naturally occurring TSP-2 polypeptide, e.g., has the ability to inhibit unwanted cell proliferation, e.g., a benign or malignant unwanted cell proliferation, e.g., a tumour growth. In one embodiment, a fragment or an analog of TSP-2 contains a type 1 repeat, e.g., a TSP-2 fragment or analog is at least 5, 10, 20, 50, 100, 150, 170, 200, or 250 amino acids in length and contains a type 1 repeat. In another embodiment, the TSP-2 fragment or analog is at least 170 amino acids in length and includes amino acids 330-500 of SEQ ID NO: 2. In a preferred embodiment the fragment is at least 50 amino acids in length and includes amino acid 330-390 of SEQ ID NO:2.

In a preferred embodiment, the nucleic acid encodes a TSP-2 protein or an encoded fragment of an analog of TSP-2 that differs in amino acid sequence at least by 1 to as many as .(but not more than) 2, 3, 5, 10, 20 or 40 residues from a sequence in SEQ ID NO:2. In a preferred embodiment, the differences, however, are such that: the nucleic acid encodes a TSP-2 protein that exhibits a TSP-2 biological activity, e.g., the encoded TSP-2 protein or a fragment or an analog thereof, retains a biological activity of a naturally occurring TSP-2 e.g., the nucleic acid encodes a the TSP-2 protein, or a fragment or an analog thereof, that can reduce the growth and size of a tumour. A difference can be a substitution, addition or deletion of a nucleic acid. In another embodiment, a TSP-2 analog can be a retro-inverso peptide, e.g., some or all of the amino acids of the sequence can be D amino acids, of a TSP-2 peptide as described herein.

In another aspect, the invention features a method of identifying active fragments or analogs of a TSP-2 polypeptide. In one embodiment, the carcinoma xenograft mouse model described herein can be used to determine if a fragment or analog can inhibit unwanted cell proliferation, e.g., inhibit tumour growth.

In another aspect, the invention features a method of making a fragment or an analog of a TSP-2 polypeptide, e.g., a TSP-2 polypeptide having at least one biological activity of a naturally occurring TSP-2 polypeptide. The method includes altering the sequence, e.g., by substitution or deletion of one or more residues, preferably which are non-conserved residues, of a TSP-2 polypeptide, and testing the altered polypeptide for the desired activity. In another preferred embodiment, the method includes altering the sequence to obtain a retro-inverso polypeptide, i.e., a polypeptide in which some or all of the amino acids are D amino acids.

In preferred embodiments the encoded TSP-2 protein includes a TSP-2 sequence described herein as well as other N-terminal and/or C-terminal amino acid sequence.

In another aspect, the invention features a vector, e.g., a cloning vector or an expression vector, containing a nucleic acid which encodes TSP-2 or a fragment or an analog thereof, e.g., a nucleic acid described herein. The vector can be a plasmid vector or a viral vector. The vector can be circular or linear. In a preferred embodiment, the vector can include one or more of the following elements, e.g., an origin of replication, a promoter, and a selection marker, e.g., a drug resistance marker. A viral vector can be a retrovirus, an adenovirus, an adeno-associated virus, an SV40 virus, or a herpes virus. Retroviral vectors are particularly useful, as they selectively integrate into the genome of replicating cells, such as tumour cells. Alternatively non-viral vectors can be used, e.g., pCDM8 (Seed (1987) Nature 329:840) and pMT2Pc (Kaufman. (1987) EMBO J. 6:187-195).

The vector can be introduced into a host cell, e.g., a bacterial cell, a yeast cell, an avian cell, or a mammalian cell, e.g., a human cell, e.g., a human epithelial cell, by standard transfection techniques, e.g., electroporation, microinjection, calcium phosphate precipitation, modified calcium phosphate precipitation, polybrene precipitation, liposome fusion, receptor-mediated DNA delivery). The vector can remain episomal, or can be incorporated into the genome of the host cell.

Another aspect of the invention features a cell that expresses TSP-2, e.g., a cell which has been genetically modified tocause the expression of a TSP-2 protein, or a fragment or an analog thereof. In one embodiment, the endogenous TSP-2 gene of the cell has been modified so as to express increased levels of the TSP-2 protein, e.g., a regulatory sequence, e.g., a promoter, or enhancer, of the TSP-2 gene has been replaced so as to express increased levels of TSP-2. In another embodiment, the cell has been manipulated, e.g., transfected or infected, with an expression vector which expresses or encodes TSP-2. The cell can be an autologous, allogeneic, or xenogeneic cell, but is preferably autologous. The autologous cell can be a cell from a subject characterized with a disorder of unwanted cell proliferation, e.g., a benign or malignant unwanted cell proliferation, e.g., a tumour The manipulated cell can be any cell type, e.g., a fibroblast, a keratinocyte, an epithelial cell, an endothelial cell, a glial cell, a neural cell, a lymphocyte, a bone marrow cell, and a muscle cell. Preferably the cell is an epithelial cell, e.g., an epidermal cell, a prostate epithelial cell, a mammary epithelial cell, or an intestinal epithelial cell. A TSP-2 nucleic acid sequence described herein can be inserted into the cell ex vivo or in vivo. If inserted ex vivo, the cell can be introduced into the subject.

In another aspect, the invention features a TSP-2 antibody. The antibody can be a polyclonal or a monoclonal antibody. The antibody can be raised, e.g., against the intact protein or a fragment thereof. In one embodiment, the antibody can bind specifically to a TSP-2 protein or a fragment. In another embodiment, the antibody binds TSP-2 with significantly greater affinity than TSP-1, e.g., 10%, 20% or 50% higher affinity. In a preferred embodiment, the TSP-2 epitope can be a 10, 15, 20 or 30 amino acid peptide of SEQ ID NO:2, e.g., the epitope is a 15-amino acid peptide DKDTTFDLFSISNIN (SEQ ID NO:3). In another preferred embodiment, the epitope can overlap the 15-amino acid peptide epitope of DKDTTFDLFSISNIN.

“Unwanted cell proliferation” refers to a cell that divides and reproduces at greater than normal levels, e.g., uncontrolled growth, e.g., a cancer cell. Unwanted cell proliferation can be benign or malignant. In one embodiment, unwanted cell proliferation can result in an abnormal mass of tissue that performs no useful function and may be deleterious to survival of the organism, e.g., a tumour. The tumour can be benign or malignant. Unwanted cell proliferation also refers to the unwanted spread of cancer cells. The spread of cancer cells can be local or peripheral. In certain instances, cancer cells can migrate (metastasis) to other parts of the body through the blood system and the lymphatic system.

A “purified” or “substantially pure” or isolated “preparation” of a polypeptide, as used herein, means a polypeptide that has been separated from other proteins, lipids, and nucleic acids with which it naturally occurs. Preferably, the polypeptide is also separated from substances, e.g., antibodies or gel matrix, e.g., polyacrylamide, which are used to purify it. Preferably, the polypeptide constitutes at least 10, 20, 50 70, 80 or 95% dry weight of the purified preparation. Preferably, the preparation contains: sufficient polypeptide to allow protein sequencing; at least 1, 10, or 100 μg of the polypeptide; at least 1, 10, or 100 mg of the polypeptide.

A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

A “treatment”, as used herein, includes any therapeutic treatment, e.g., the administration of a therapeutic agent or substance, e.g., a drug.

As used herein, the term “subject” refers an animal, e.g., a mammal, e.g., a human. The mammal can be a human or non-human mammal, e.g., a swine, a bird, a cat, a dog, a monkey, a goat, or a rodent, e.g., a rat or a mouse.

An “isolated” or “pure nucleic acid”, e.g., a substantially pure DNA, is a nucleic acid which is one or both of: not immediately contiguous with either one or both of the sequences, with which it is immediately contiguous (i.e., one at the 5′ end and one at the 3′ end) in the naturally-occurring genome of the organism from which the nucleic acid is derived; or which is substantially free of a nucleic acid sequence with which it occurs in the organism from which the nucleic acid is derived. The term includes, for example, a recombinant DNA which is incorporated into a vector, e.g., into an autonomously replicating plasmid or virus, or into the genomic DNA of a prokaryote or eukaryote, or which exists as a separate molecule (e.g., a cDNA or a genomic DNA fragment produced by PCR or restriction endonuclease treatment) independent of other DNA sequences. Substantially pure DNA can also include a recombinant DNA which is part of a hybrid gene encoding sequence.

“Regulatory sequence” refers to any or all of the DNA sequences that controls gene expression. An example of a regulatory sequence includes: a promoter, a positive regulatory element (such as an enhancer or a DNA-binding site for a transcriptional activator); a negative regulatory element (such as a DNA-binding site for a transcriptional repressor) and an insulator.

“Heterologous” refers to DNA or tissue which is derived from a different species.

“Heterologous regulatory sequence” refers to a sequence which is not the normal regulatory sequence of that gene.

“Sequence identity or homology”, as used herein, refers to the sequence similarity between two polypeptide molecules or between two nucleic acid molecules. To determine the percent homology of two amino acid sequences (e.g., SEQ ID NO:2) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., SEQ ID NO:2) is occupied by the same amino acid residue or nucleotide as the corresponding position in the other sequence, then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100). For example, if 6 of 10, of the positions in two sequences are matched or homologous then the two sequences are 60% homologous or have 60% sequence identity. By way of example, the DNA sequences ATTGCC and TATGGC share 50% homology or sequence identity. Generally, a comparison is made when two sequences are aligned to give maximum homology or sequence identity.

The comparison of sequences and determination of percent homology between two sequences can be accomplished using a mathematical algorithm. A preferred, non-limiting example of a mathematical algorithm utilized for the comparison of sequences is the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to TSP-2 nucleic acid molecules of the invention. BLAST protein searches can be performed with the XBLAST program, score=50, wordlength=3 to obtain amino acid sequences homologous to TSP-2 protein molecules of the invention. To obtain gapped alignments for comparison purposes. Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The terms “peptides”, “proteins”, and “polypeptides” are used interchangeably herein.

The term “small molecule”, as used herein, includes peptides, peptidomimetics, or non-peptidic compounds, such as organic molecules, having a molecular weight less than 2,000, preferably less than 1,000.

A polypeptide has TSP-2 biological activity if it has one or more of the properties of TSP-2 disclosed herein, e.g., it can decrease tumour size or decrease vascularity in the in vivo mouse model described herein. A polypeptide has biological activity if it is an antagonist, agonist, or super-agonist of a polypeptide having one of the properties of TSP-2 disclosed herein.

“Misexpression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus. As described herein, one aspect of the invention features a substantially pure (or recombinant) nucleic acid which includes a nucleotide sequence encoding a TSP-2 polypeptide and/or equivalents of such nucleic acids. The term nucleic acid as used herein can include fragments and equivalents. The term equivalent refers to nucleotide sequences encoding functionally equivalent polypeptides. Equivalent nucleotide sequences will include sequences that differ by one or more nucleotide substitutions, additions or deletions, such as allelic variants, and include sequences that differ from the nucleotide sequences disclosed herein by degeneracy of the genetic code.

As used herein, the term “hybridizes under stringent conditions” refers to conditions for hybridization and washing under which nucleotide sequences typically remain hybridized to each other. Such stringent conditions are known to those skilled in the art and can be found in Current Protocols in Molecular Biology, John Wiley & Sons, New York (1989), 6.3.1-6.3.6. A preferred example of stringent hybridization conditions are hybridization in 6X sodium chloride/sodium citrate (SSC) at about 45° C., followed by one or more washes in 0.2×SSC, 0. 1% SDS at 50-65° C. Preferably, an isolated nucleic acid molecule of the invention that hybridizes under stringent conditions to the sequence of SEQ ID NO:1 corresponds to a naturally occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural TSP-2 protein.

Other features and advantages of the invention will be apparent from the following detailed description, and from the claims.

### BRIEF DESCRIPTION OF THE DRAWINGS

FIG. 1A-B depicts the nucleotide sequence of TSP-2 (SEQ ID NO: 1).

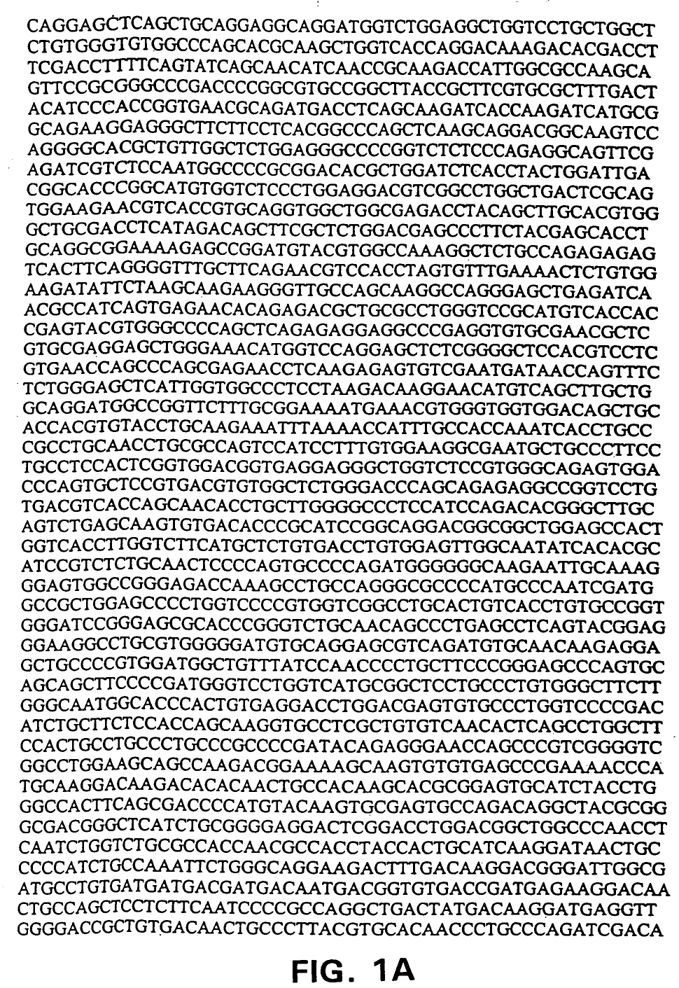

FIG. 2 depicts and the amino acid sequence of TSP-2 (SEQ ID NO:2).

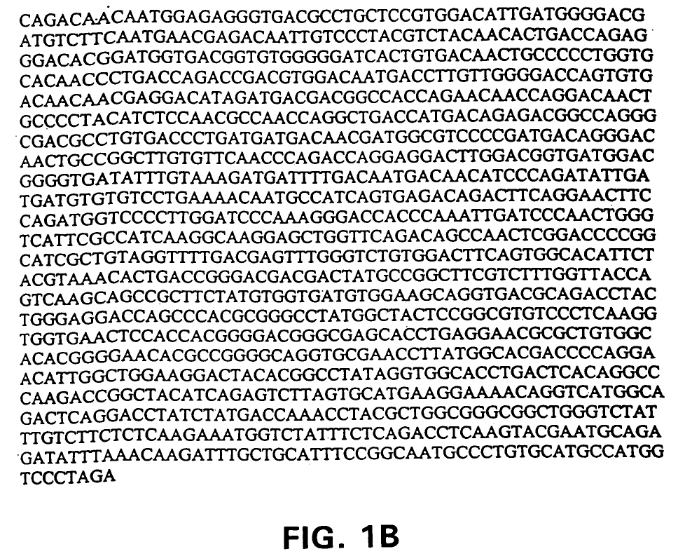

FIG. 3 is a graph showing that transfected TSP-2 inhibits intradermal tumor growth of A431 squamous cell carcinoma cells (left) and MeWo malignant melanomas (right).

FIG. 4 depicts graphs showing the effects of TSP-2 on tumor angiogenesis, the average vessel density (Panel A), vessel size (Panel B), the number of vessels found in the size range of less than 500 μm² and larger than 1500 μm²(Panel C), and the percentage of tissue area covered by vessels (Panel D).

FIG. 5 shows the development and incidence of papillomas in TSP-2 deficient mice treated with a DMBA/TPA chemical carcinogenic protocol. Panel A shows accelerated development of papillomas in TSP-2 deficient mice. Panel B shows a highly increased incident of papillomas in TSP-2 deficient mice.

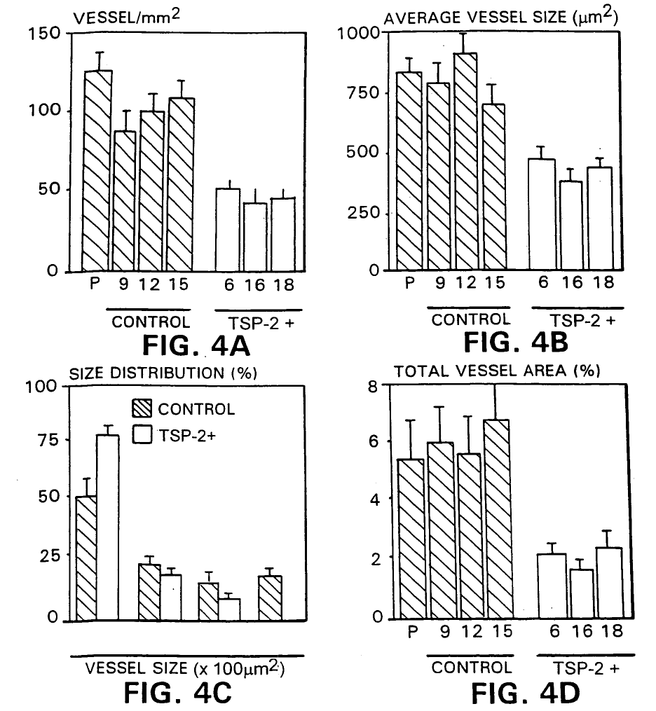

FIG. 6 is a graph showing the migration of human dermal microvascular endothelial cells (HDMEC) and the effect of TSP-1 (T1) or TSP-2 (T2) binding of the CD36 receptor on the migration of these cells. HDMEC were incubated alone (C), in the presence of TSP-1 (T1) or TSP-2 (T2), or in the presence of TSP-1 (T1) or TSP-2 (T2) in the presence of an anti-CD36 antibody (36).

FIG. 7 is a graph showing the effect of HDMEC migration in the presence of various synthetic TSP-2 derived peptides. Peptides 1, 2, 3 and 4 (P1, P2, P3, P4) were derived from the procollagen domain of TSP-2, peptide 7 (P7) was derived from the first type 1 repeat of TSP-2.

### DETAILED DESCRIPTION

nThe present invention is based, in part, on the discovery that TSP-2 molecules can be used to treat unwanted angiogenesis and cell proliferation, e.g., inhibit tumour growth.

Production of an Anti-TSP-2 Antibody

A15-amino acid peptide DKDTTFDLFSISNIN (SEQ ID NO:3), derived from the N-terminal sequence of the TSP-2 coding region (AA 22-36) was used to immunize two rabbits using standard techniques. The sequence begins shortly after the end of the signal sequence, and has only three amino acids in common with human TSP-1. This sequence was chosen to increase the likelihood that the antisera would not cross-react with human TSP-1.

A polyclonal antibody, R81939, was obtained which specifically detected two bands of approximately 180 and 135 kDa in Western blots of endothelial cell, keratinocyte, fibroblast, and endothelial cell lysates and conditioned media, corresponding to TSP-2. The specificity of the antibody was demonstrated by the lack of detection of natural human TSP-1, purified from human platelets. This antibody was affinity purified, using the identical 15-AA peptide, and was used for immunohistochemical analyses. The specificity of the antibody was further demonstrated by enhanced detection of TSP-2 protein obtained from the conditioned media of TSP-2 transfected A431 cells using antibody R81939, while there was an absence of TSP-2 in conditioned media obtained from vector only transfected A431 clones and from TSP-1 overexpressing clones. Thus, R81939 selectively recognized secreted TSP-2 but not TSP-1 in media conditioned by transfected A431 tumor cells.

Cloning of Human TSP-2 cDNA

PCR amplification was performed on human TSP-2 cDNA, using Marathon-Ready cDNA obtained from human placenta (Clontech, Palo Alto, Calif.) and the human TSP-2 specific primers 5′-GAATTCAGGAGCTCAGCTGCAGGAGGC-3′ (SEQ ID NO:4) (forward primer) and 5′-GAATTCTAGGGACCATGGCATGCAC-3′ (SEQ ID NO:5). PCR was performed using the Expand Long Template PCR System (Boehringer-Mannheim, Mannheim, Germany) according to the manufacturer's instructions. PCR conditions were as follows: incubation at 94° C. for 2 minutes, followed by 10 cycles with each 10 seconds at 94° C., 30 seconds at 65° C, and 2 minutes at 68° C. Another 25 cy followed with identical incubation temperatures and times, except that the incubation at 68° C. was extended by additional 20 seconds for each additional cycle. The 3.6-kb PCR product was visualized on a 1% agarose/1×TAE gel, and was gel purified, using the QIAEX kit (Qiagen) according to the manufacturer's instructions. It was then polished and cloned into the EcoRI restriction site of the pCR-Script cloning vector (Invitrogen) according to the manufacturer's instructions. The successful insertion into the cloning vector was confirmed by restriction mapping and by direct sequencing using the Sanger dideoxy method. Four distinct cDNA clones were completely sequenced. Clone 10 consists of 3,596 bp of specific human TSP-2 sequence, including the complete coding sequence (from nucleotide 26 to nucleotide 3544). The cDNA sequence shows 99.6% identity with the GenBank accession number L12350 (LaBell et al., Genomics, 1992, 12:421-429). The deducted amino acid sequence of clone 10 comprises 1172 amino acids and shows a 99.6% similarity with the deducted amino acid sequence of L12350.

The TSP-2 cDNA was then cloned into the pSecTag vector (Invitrogen), and was used to transfect insect cells. Recombinant human TSP-2, secreted into the culture media, was purified by heparin-Sepharose columns and, under reduced conditions, was found in two forms, as the 180-kDa intact molecule and as a 135-kDa cleavage product.

Using these methods, full-length TSP-2 has been obtained but the protein yields have been relatively low. Therefore, 293 human embryonic kidney cells were transfected with a different human TSP-2 expression vector. A PCEP4 vector (Invitrogen) was used that was modified as follows: a BM 40 signal peptide sequence was introduced in front of the insertion site of TSP-2, the antibiotic selection gene was replaced with a puromycin gene for fast and efficient antibiotic selection of stably transfected clones, and a total of 8 histidin residues at the C-terminal end have been included to facilitate purification of the recombinant protein. Using this vector, stably transfected 293 cells produce high amounts of the recombinant protein and the use of mammalian cells ensures efficient glycosylation of recombinant TSP-2. Four different recombinant TSP-2 proteins have now been expressed. Construct I expresses selectively the N-terminal procollagen domain of TSP-2 (nucleotides 294-1367), the region with the least homology to TSP-1. Construct 2 expresses, in addition, the type I repeats (nucleotides 294-1883) which contain several biologically active sites including two CSVTCG sequences that mediate binding to the CD36 receptor on endothelial cells. Construct 3 expresses the type I repeats (nucleotides 1383-1883) only. Construct 4 expresses the full-length mature TSP-2 molecule, excluding the signal peptide (nucleotides 294-3755) which is provided in the expression vector. Such recombinant proteins can be used for the generation of monoclonal anti-TSP-2 antibodies, for the establishment of a human TSP-2 ELISA, and for the systemic treatment of experimental tumors.

Cell Culture

The human epidermoid carcinoma cell line A431 was obtained from the American Type Culture Collection (Rockville, Md.), and was maintained in Dulbecco's minimal essential medium (DMEM; Gibco BRL, Grand Island, N.Y.) supplemented with 10% fetal bovine serum (FBS) and 1% L-glutamine (all purchased from Gibco BRL). Human dermal microvascular endothelial cells (HDMEC) were isolated from neonatal foreskins and cultivated as recently described by Richard et al., Exp Cell Research 1998, 240:1-6. Normal human prostate epithelial cells were purchased from Clonetics, and human PC-3 prostate cancer cells were obtained from the American Type Culture Collection.

Isolation of TSP-2 RNA and Northern Blot Analysis

Total cellular RNA was isolated from normal prostate epithelial cells, from PC-3 prostate cancer cells, from stable A431 cell transfectants and from intradermal tumors using the RNeasy kit (Qiagen), according to the manufacturer's instructions. The isolated RNA was subjected to electrophoresis and transferred to Biotrans nylon supported membranes (ICN Pharmaceuticals, Costa Mesa, Calif.). 32P-radiolabeled cDNA probes were prepared with a random primed synthesis kit (Multiprime; Amersham, Arlington Heights, Ill.). A 3.6 kb TSP-2 cDNA probe, a 4.1 kb TSP-1 cDNA probe and a 300-bp human VEGF cDNA-probe which recognizes all known VEGF variants. A 2.0 kb human β-actin cDNA probe purchased from Clontech (Palo Alto, Calif.) was used as a control for equal RNA loading. Blots were washed at high stringency as described in Detmar et al. (J Invest Dermatol 1997, 108:263-268), and exposed to X-OMAT MR film (Kodak, Rochester, N.Y.) or a Phosphor Imager screen (Molecular Dynamics, Sunnyvale, Calif.). mRNA expression was quantitated with a Molecular Dynamics scanning densitometer, using the ImageQuant software. Total cellular RNA was isolated from stable transfectants and from intradermal tumors using the RNeasy kit (Qiagen), according to the manufacturer's instructions. The isolated RNA was subjected to electrophoresis and transferred to Biotrans nylon supported membranes (ICN Pharmaceuticals, Costa Mesa, Calif.). ³²P-radiolabeled cDNA probes were prepared with a random primed synthesis kit (Multiprime; Amersham, Arlington Heights, Ill.). A 4.1 kb TSP-1 cDNA probe and a 300-bp human VEGF cDNA-probe which recognizes all known VEGF variants as used. A 2.0 kb human β-actin cDNA probe purchased from Clontech (Palo Alto, Calif.) was used as a control for equal RNA loading. Blots were washed at high stringency as described by Detmar et al. (J. Invest Dermatol, 1997, 108:263-268) and exposed to X-OMAT MR film (Kodak, Rochester, N.Y.) or a Phosphor Imager screen (Molecular Dynamics, Sunnyvale, Calif.). mRNA expression was quantitated with a Molecular Dynamics scanning densitometer, using the ImageQuant software.

Western Blot Analysis

Western Blot analyses were performed on cell lysates and conditioned media from stably transfected A431 and HDMEC. Cells were grown to confluence in 100 mm dishes, washed with phosphate buffered saline (PBS) and lysed as described by Gallop et al. (J Med Chem 1994, 37:1233-1251). Cell lysates were homogenized using a cell shredder (Qiagen), and protein concentrations were determined using the Bio-Rad protein assay (Bio-Rad, Hercules, Calif.). Conditioned media were obtained from confluent cells grown for 48 hours in serum-free culture medium. TSP-2 was concentrated using heparin beads (Sigma, St Louis, Mo.). All samples were boiled in de-naturating sample buffer, and equal amounts according to the protein assay were electrophoresed on polyacrylamide gels under reducing conditions (Laemmli, Nature 1970, 227:680-685). Proteins were blotted onto polyvinylidene difluoride membranes (Bio-Rad). To verify equal protein loading, membranes were stained with 0.1% Ponceau red (Sigma) diluted in 5% acetic acid. Membranes were incubated overnight in PBS containing 0.1% Tween-20 and 3% bovine serum albumin to block nonspecific binding. Membranes were then incubated with primary antibodies directed against TSP-2 (clone R81939), human TSP-1 (clone 133; Genzyme, Cambridge, Mass.), washed in PBS/Tween, incubated with horseradish peroxidase-conjugated anti-mouse IgG (Amersham), and analyzed by the enhanced-chemiluminescence system (Amersham). Protein expression was quantitated with a Molecular Dynamics scanning densitometer, using the ImageQuant software.

Analysis of TSP-2 Expression in Prostate Cancer Cells

nNormal human prostate epithelial cells and the malignant human prostate cancer cell line PC-3 were analyzed for their expression of TSP-2 mRNA using Northern blot analysis. These studies demonstrated that normal prostate cells strongly produce TSP-2, whereas TSP-2 expression was completely absent in PC-3 cells. These data suggest loss of TSP-2 expression as an important step in the pathogenesis of malignant prostate cancer, similar to our findings in squamous cell carcinomas, and indicate an important role of TSP-2 in the control of prostate cancer growth and tumor angiogenesis. In addition, an absence of TSP-2 expression in PC-3 cells was found after orthotopical intraprostatic injection in vivo.

Analysis of TSP-2 Expression in Squamous Cell Carcinomas

Immunohistochemistry studies with anti-TSP-2 antibody R81939 demonstrated strong TSP-2 expression in basal epidermal keratinocytes of healthy adult human skin of a patient with squamous cell carcinoma (SCC) and of neonatal human foreskin. In addition, TSP-2 was deposited in the basement membrane area. These findings suggest that TSP-2 contributes to the natural anti-angiogenic barrier in the skin, preventing ingrowth of blood vessels into the non-vascularized epidermis. Moreover, TSP-2 might also contribute to the maintenance of normal epidermal architecture. In contrast, TSP-2 expression was absent in the basal epidermal layer in the hyperproliferative epidermis in close vicinity to SCC, but was diffusely present in suprabasal layers. TSP-2 expression was greatly reduced in 4 out of 4 examined human SCC with different grades of malignancy. No deposits of TSP-2 surrounding the tumor cells were detected, and invasive tumor cells did not express TSP-2. These results were confirmed by in situ hybridization, using a human TSP-2 antisense riboprobe, and suggest that decreased expression of TSP-2 in SCCs may diminish the endogenous anti-angiogenic barrier and may facilitate tumor angiogenesis, growth and invasion.

Production of TSP-2 Transfected Tumor Cell Lines

A 3.6 kb mouse TSP-2 cDNA sequence, comprising the full TSP-2 coding sequence, was provided by Dr. Paul Bornstein, University of Washington, Seattle. A 3.6 kb EcoRI-mTSP-2 fragment was cloned into EcoRI-site of the PIRES/Neo vector (Clonetics, Palo Alto, Calif.). Subconfluent A431 cell cultures were stably transfected either with PIRES/Neo vector containing the full-length mouse TSP-2 cDNA or with PIRES/Neo vector alone using the SuperFect transfection reagent (Qiagen, Chatsworth, Calif.) according to the manufacturer's protocol. In addition, A431 cells that had previously been transfected with a pcDNA3.1/Zeo+ expression vector containing the human TSP-1 gene were transfected. Transfections were performed with the calcium phosphate method, and drug selection was achieved by culturing the transfected cells in the presence of G418. In particular, forty-eight hours after transfection, cells were split 1:3 into their full growth medium containing 400 mg/ml Neomycin (G418, Sigma, St Louis, Mo.) to select transfectants. Stably transfected clones were expanded, and 10 clones were characterized for TSP-1 MRNA and protein expression. More than 10 stably transfected clones were obtained for each construct and confirmed efficient TSP-2 expression by Northern hybridization and by Western blotting of cell lysates and conditioned media. Importantly, TSP-2 was virtually absent in conditioned media obtained from confluent control A431 cultures transfected with vector only. No significant differences in cellular morphology and growth rates on plastic culture dishes, in soft agar colonization or in spontaneous and induced apoptosis rates were observed between control transfected and TSP-2 overexpressing A431 clones. In addition, TSP-2 overexpressing MeWo malignant melanoma cells and PC-3 prostate carcinoma cells were established and characterized.

The human squamous cell carcinoma cell line A431 is characterized by strong secretion of VEGF but little or no TSP-2 secretion and forms fast growing and highly vascularized tumors in vivo (Myoken et al., Proc Natl Acad Sci USA 1991, 88:5819-5823). The TSP-2 expression levels of multiple TSP-2 and control transfected clones were determined by Northern blot analyses. High levels of TSP-2 mRNA were detected in A431 clones 6, 12, and 19 which were used for further in vivo studies. In addition, these clones did not show down regulation of VEGF mRNA expression. Western blot analyses confirmed that increased TSP-2 mRNA levels correlated with increased amounts of TSP-2 protein. In TSP-2 transfected A431 cell clones, strong expression of the 180 kd TSP-2 protein was found in culture supernatants, confirming efficient secretion of TSP-2. In contrast, little or no TSP-2 protein was detected in A431 cells transfected with vector only.

Analysis of TSP-2 Overexpression on Cell Growth and Apoptosis In Vitro

To determine whether TSP-2 overexpression influences tumor cell proliferation, anchorage-independent cell growth rates were measured as described Schirner et al. (Clin Exp Metastasis, 1998, 16:427-435). Ten thousand control transfected or TSP-2 transfected A431 cells were transferred into six 30 mm cell culture dishes with 2 mm grid (Nunc, Naperville, Ill.). The dishes were incubated at 37° C. and 5% CO2, and colonies were counted after 8 days. The results represent the mean values ± standard deviation (SD) of four dishes per group.

Anchorage-independent cell growth was studied by determination of colony numbers in a soft agar assay. No significant differences in the number of colonies were observed between TSP-2 overexpressing and control cell clones.

Analysis of TSP-2 Expressing Xenografts In Nude Mice

To determine the biological effects of TSP-2 overexpression on the orthotopic tumor growth of A431 cells in vivo, tumor cells were injected intradermally into the flanks of immunodeficient nude mice.

Confluent A431 cells, untransfected or stably transfected with the mouse TSP-2 expression vector, a human TSP-1 expression vector or with the expression vector alone, were trypsinized and resuspended in serum-free DMEM medium (Gibco BRL) at a density of 2×10⁷ cells/ml. Two million tumor cells of each type were injected intradermally into both flanks of five 8 weeks old female Balb/C (nu/nu) mice. The parental A431 cell line, three control clones, three TSP-2 overexpressing cell clones and three TSP-1 overexpressing clones were investigated. In particular, mice were injected with cells from three TSP-2 transfected clones, three vector-transfected control clones, and the maternal A431 cell line. For comparison of TSP-2 effects with the previously described effects of TSP-1, mice were also injected with three TSP-1 transfected clones and with three clones of A431 cells that were transfected with both TSP-1 and TSP-2. The smallest and largest tumor diameter were measured weekly, using a digital caliper, and tumor volumes were calculated using the following formula:
Volume=4/3×p×(½×smaller diameter)²×½×larger diameter.
Mice were sacrificed after 3 weeks in the group of animals injected with parental cells, control or TSP-1 transfected cells. Three out of five animals injected with TSP-2 overexpressing A431 clones were sacrificed after 3 weeks and two animals after 6 weeks.

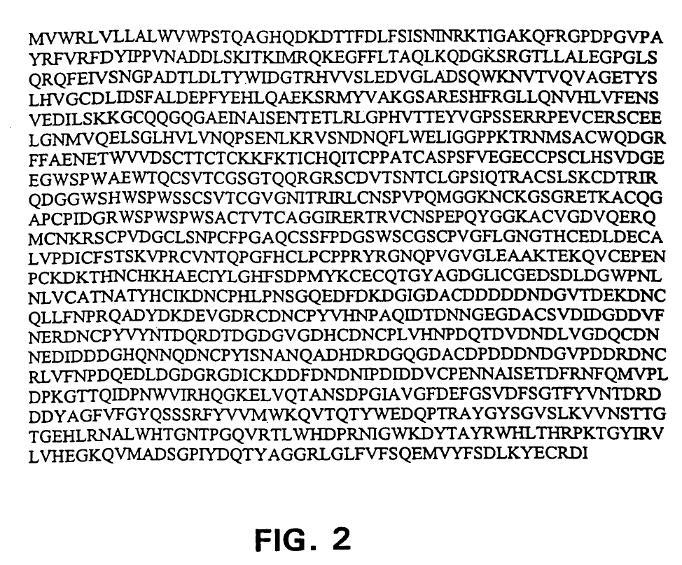

As shown in FIG. 3 (left), control A431 and vector-transfected cell clones formed rapidly growing squamous cell carcinoma, reaching a volume of 2000-3000 mm³ after 3 weeks. Overexpression of TSP-2 resulted in a significant inhibition of tumor growth by more than 90% (p<0.001) after 3 weeks, as compared to control tumors. The TSP-2 induced inhibition of squamous cell carcinoma growth was significantly more potent than the 40-50% inhibition observed in TSP-1 expressing tumors (p<0.001). Importantly, none of the three clones co-transfected with both TSP-2 and TSP-1 formed any visible tumors over an observation period of up to 12 weeks. TSP-2 overexpression also decreased tumor angiogenesis, as shown by a decreased density of tumor vessels, as compared to control tumors. As shown in FIG. 3 (right), similar results were obtained using the human malignant melanoma cell line MeWo.

In situ Hybridization and Immunohistochemistry from Mice Having Xenografts Which Express TSP-2

In situ hybridization was performed on 6 μm paraffin sections of tumor xenografts as described by Gallop et al., supra. The sense and antisense single-stranded RNA-probes for human VEGF were transcribed from a pGEM-3Zf(+) vector containing a 204 bp PCR fragment common to all known VEGF splicing variants. A RNA-probe to murine TSP-2 was transcribed from a pBluescript II KS+ vector containing a 350-bp PCR fragment of the amino terminal coding region of human TSP-2. Immunohistochemical stainings were performed on 6 μm frozen or paraffin sections of normal adult human skin, normal neonatal human foreskin, human squamous cell carcinomas of the skin and A431 tumor xenotransplants as previously described by Detmar et al., (J Invest Dermatol 1998, 111:1-6), using a monoclonal antibody against human TSP-1 (Genzyme) and rabbit polyclonal antibody R81939 against human TSP-2. R81939 recognizes both human and mouse TSP-2.

Extensive areas of necrosis were detected in TSP-2 overexpressing tumors, whereas only small necrotic foci were found in control tumors, and less necroses were found in TSP-1 overexpressing tumors. Little or no TSP-2 mRNA expression was detected in control tumor cells, and TSP-2 protein expression was predominantly found in the basal epidermal layer of adjacent normal skin and in blood vessels, but not in tumor cells. In contrast, strong TSP-2 mRNA expression was detected in TSP-2 overexpressing tumor cells, and immunohistochemistry demonstrated massive TSP-2 expression in tumor cells and in the tumor stroma. No differences of VEGF mRNA expression were found between TSP-2 overexpressing and control tumors by in situ hybridization. Similar results were obtained using the human malignant melanoma cell line MeWo.

Role of TSP-2 Overexpression in Tumor Angiogenesis

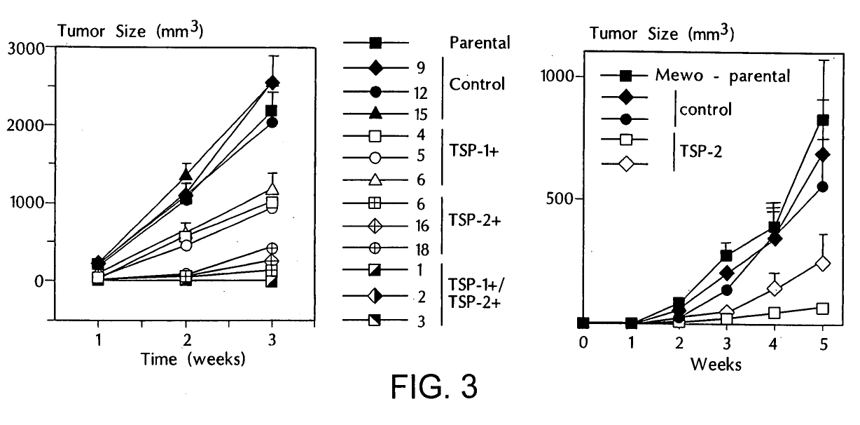

To determine the degree of tumor-induced angiogenesis, cryostat sections of tumor xenografts were stained with a rat monoclonal anti-mouse CD31 antibody (Pharmingen, San Diego, Calif.). Representative sections obtained from five tumors from each cell clone were analyzed, using a Nikon E-600 microscope (Nikon; Melville, N.Y.). Images were captured with a Spot digital camera (Diagnostic Instruments; Sterling Heights, Mich.), and morphometric analyses were performed using the IP LAB software program (Scanalytics Inc.; Fairfax, Va.). Three different fields at 60× magnification were examined on each section, and the number of vessels per mm² was determined. Morphometric analysis revealed highly decreased microvessel densities in 3 weeks-old tumors derived from TSP-2 overexpressing clones T6 (46±15 vessels/mm²), T16 (39±14 vessels/mm²), and T18 (41±14 vessels/mm²), as compared to control clones C9 (84±22 vessels/mm²), C12 (98±27 vessels/mm²) and C15 (105±19 vessels/mm²). These sections demonstrated a dramatic reduction of microvessels within TSP-2 expressing tumors. To achieve a more detailed quantification of the effects of TSP-2 on tumor angiogenesis, the average vessel density, vessel size, and percentage of tissue area covered by vessels were determined by computer-assisted image analysis of representative digital images as previously described in Detmar et al. (2000) Am. J. Pathol. 156:159-167 and Streit et al. (1999) Proc. Natl Acad. Sci. USA 96:14888-14893. While control tumors showed between 80 and 125 CD31 positive vessels per mm² tumor area, as shown in FIG. 4A, the vascular density was reduced by more than 50% in TSP-2 expressing tumors. Moreover, the average vessel size was reduced by more than 45% in TSP-2 overexpressing tumors (see FIG. 4B). In particular, as shown in FIG. 4C, TSP-2 expression resulted in complete absence of blood vessels larger than 1500 μm² which represented 15% of all blood vessels in control tumors. In accordance with these data, the relative tumor area occupied by vessels was reduced by 70% in TSP-2 transfected tumors (p<0.001) (see FIG. 4D). These data demonstrate that overexpression of TSP-2 in experimental squamous cell carcinomas potently inhibited tumor angiogenesis.

Development of Skin Papillomas and Squamous Cell Carcinoma in TSP-2 Deficient Mice

Breeding pairs of TSP-2 deficient mice were obtained from Dr. Paul Bornstein, Seattle. The construction of the targeting vector and the generation of TSP-2 deficient mice on a homogenous 129Sv genetic background have been previously described in Kyriakides et al. (1998) J. Cell Biol. 140:419-430. TSP-2 deficient mice show an increased density of blood vessels in several organs including the skin.

Because TSP-2 is expressed in basal epidermal keratinocytes but not in SCC, and because overexpression of TSP-2 inhibits SCC growth, TSP-2 expression in the skin might play a protective role against skin tumor development. Thus, a standard, two-stage skin carcinogenesis protocol, as described in Hennings et al. (1981) Cancer Res. 41:773-779 and Hennings et al. (1993) Carcinogenesis 14:2353-2358, was performed in 25 female TSP-2 deficient mice and in 25 age-matched female wildtype mice. Topical application of 25 μg DMBA, dissolved in 200 μl acetone, was applied to the shaved back of 8-weeks-old mice, followed by 20 weekly applications of 5 μg TPA. A significantly earlier development of papilloma formation was found in TSP-2 deficient mice (50% of mice were tumor-bearing after 8 weeks vs. 14 weeks for wildtype controls). Moreover, TSP-2 deficient mice developed highly increased numbers of papillomas (more than nineteen per mouse after 20 weeks), as compared to wildtype mice (less than five per mouse). These papillomas were also larger and better vascularized than in wildtype mice. Importantly, TSP-2 deficient mice also developed increased numbers of squamous cell carcinomas. These results reveal a protective role of TSP-2 against chemical skin carcinogenesis.

Production of Transgenic Mice Overexpressing TSP-2 Selectively in Skin.

A 4.1-kb mouse TSP-2 cDNA sequence, comprising the full TSP-2 coding sequence, was cloned into a pBluescript II KS vector. After restriction digestion with SalI and Xbal, the 4.1 kb fragment was gel purified, blunted, and ligated into a blunted, BamHI digested pGEM-3Z vector containing the human keratin 14 (K14) promoter that has been shown previously to target transgene expression to the skin. K14 is predominantly expressed by basal keratinocytes in the epidermis and outer root sheath of hair follicles in the skin, although some expression has been reported in the esophagus, forestomach, squamous islets of the thymus, and the cornea. The correct sequence and orientation of the TSP-2 insert were verified by direct sequencing using the Sanger dideoxy method.

After digestion of the complete 11.5-kb K14-TSP-2-pGEM3Z construct described above with the restriction enzymes Kpnl and HindlIl, the 8.3-kb expression vector was gel purified and injected into the female pronucleus of FVB/N mouse embryos at the one-cell stage. After overnight culture, embryos (now at the two-cell stage) were injected into the uterus of pseudo-pregnant mice. Transgenic founders were detected by Southern blot analysis of BamHI digested genomic tail DNA obtained 2 weeks after birth, using a ³²P-labeled 350-bp mouse TSP-2 cDNA as a probe. For rapid identification, genomic tail DNA was subjected to PCR using an 18-mer primer and a 21-mer primer that bind, respectively, to positions 321-338 and 650-630 of the human growth hormone gene in the transgene construct, leading to selective amplification of a 330 bp fragment if the transgene construct was incorporated into the genome. 12 viable founders transgenic for TSP-2 with different levels of transgene expression were obtained.

All transgenic founders have been backcrossed with wildtype mice. 11 founders were fertile, and 8 transmitted the transgene to their offspring. Three founders with copy numbers between approximately 10 and approximately 20 as determined by Southern blot and phosphorimager quantitation were chosen for the generation of transgenic lines. Heterozygote F1-generation mice were crossed, and homozygous and heterozygous transgenic F2-generation mice and wildtype F2-generation mice were obtained. Transgene expression has been confirmed by in situ hybridization and by Northern hybridization of total RNA extracted from the skin. Immunohistochemistry of tissue sections obtained from the tails of transgenic TSP-2 founders at 2 weeks after birth confirmed TSP-2 overexpression, predominantly deposited close to basal keratinocytes of the epidermis and around hair follicles. On routine H&E-stained tissue sections, no major skin abnormalities were detected. However, staining for the endothelial cell specific antigen CD31 (PECAM-1) demonstrated decreased microvascular density in the skin of TSP-2 overexpressing transgenic mice, as compared to their littermate controls.

Production of TSP-2 Overexpressing Human Dermal Fibroblasts

PT67-packaging cells were grown in complete DMEM to 70% confluence and were transfected with the pLXSN vector (Clontech) alone or with a pLXSN vector containing the complete coding sequence of the human TSP-2 gene. After antibiotic selection with 800 1μg/ml G418, approximately 50 clones were expanded and viral titers were determined, using serial dilutions of filtered culture supernatants and G418-treated NIH 3T3 cells as described by the manufacturer. Viral titers of at least 1×10⁶ were considered to be sufficient for further use. In a next step, filtered culture supernatants with high viral titers, obtained from PT67 packaging cells, were used to transfect IMR91 fibroblasts. The efficiency of infection was assessed by G418 antibiotic selection. Transfected cells were grown to confluence, the medium was changed, and cells were cultured for an additional 48 hours. Cellular RNA was extracted, using the Qiagen Rneasy kit, and was processed for Northern blots. Culture supernatants were used for Western blot analyses. High levels of TSP-2 mRNA expression and efficient TSP-2 secretion by transfected fibroblasts was obtained.

Determination of the Effect of TSP-2 Binding of the CD36 Receptor On Migration of Endothelial Cells

To determine the effect of TSP-1 or TSP-2 binding of the CD36 receptor on the migration of human dermal microvascular endothelial cells (HDMEC), HDMEC were incubated alone, in the presence of TSP-1 or TSP-2, or in the presence of TSP-1 or TSP-2 and an anti-CD36 antibody. Briefly, eight μm pore size Transwell migration chambers (Costar, Cambridge, Calif.) were coated on the underside with 10 μg/ml collagen type I (Nalgene, Palo Alto, Calif.). 1×10⁵ HDMEC were added to the upper chamber in 300 μl of DMEM medium, or in DMEM medium containing 10 μg/ml human thrombospondin-1 (TSP-1), or in conditioned medium obtained from control transfected A431 clones (CM-Co) or from TSP-2 transfected A431 clones. All media were supplemented with 10 mg/ml BSA. Media were also supplemented either with 10 μg/ml control IgG (IgG) or with 10 μg/ml anti-CD36 antibody (clone FA6-152, Immunotech). After 4 h, migrated cells were fixed and stained as previously described Senger et al. (1996) Am. J. Pathol. 49:293-305. Images of three different 10× fields were captured from each membrane with a Spot digital camera (Diagnostic Instruments; Sterling Heights, Mich.) attached to a Nikon E-600 microscope (Nikon; Melville, N.Y.) and the number of migrating cells was calculated per mm², using the IP-LAB software (Scanalytics, Fairfax, Va.). All assays were performed in quadruplicate.

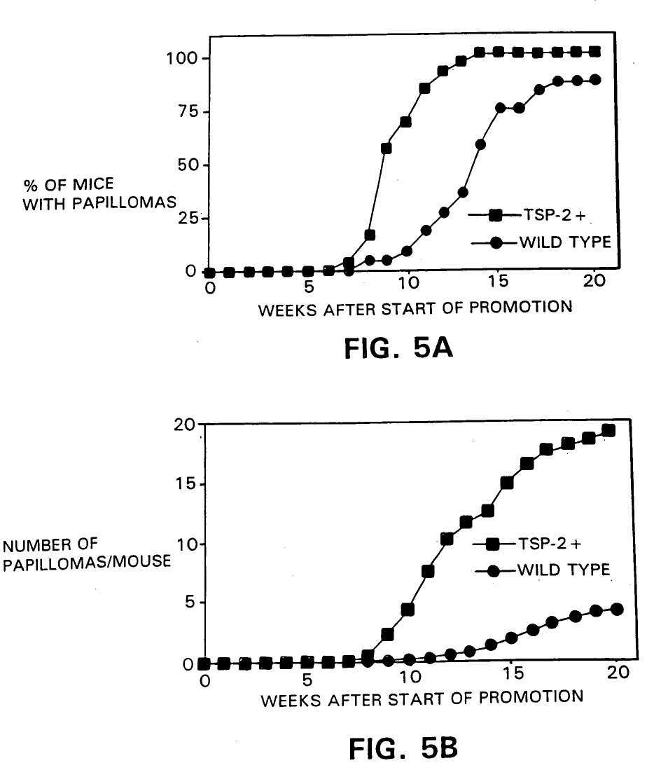

As shown in FIG. 6, in DMEM medium, 226±52 HDMEC/mm² migrated to the underside of the inserts (column 1). TSP-1 inhibited HDMEC migration by 54% (104±20 HDMEC/mm², column 2). In the presence of an anti-CD36 antibody, TSP-1 inhibited HDMEC migration by only 20.8% (column 3; 179±36 HDMEC/mm²). This shows that most of the inhibitory effect of TSP-1 was mediated through interaction with the CD36 receptor on HDMEC.

In control-conditioned medium (obtained from control-transfected A431 cells), 120±4 HDMEC/mm² migrated to the underside of the inserts (column 4). TSP-2 conditioned medium potently inhibited HDMEC migration by 54.2% (55±9 HDMEC/mm², column 5). Addition of the anti-CD36 antibody did not inhibit HDMEC migration (133±27 HDMEC/mm², column 6). In the presence of an anti-CD36 antibody, TSP-2 conditioned medium still inhibited HDMEC migration by 36.1% (85±4 HDMEC/mm², column 7). These data demonstrate that most of the TSP-2 mediated inhibitory effect was independent of interaction with the CD36 receptor on HDMEC.

Synthetic TSP-2 Derived Peptides

The following synthetic peptides, derived from the amino acid sequence of human TSP-2, were synthesized:
Peptide 1: RESHFRGLLQNVHLVF: procollagen domain, AA 207-222
Peptide 2: PATCANPSFVEGECCPSC: procollagen domain, AA 366-383
Peptide 3: FAENETWVVDSCTTCTCKKFKT: procollagen domain, AA 336-357
Peptide 4: ELIGGPPKTRNMSAC: procollagen domain, AA 315-329
Peptide 7: WSPWAEW: first type I repeat, AA384-390

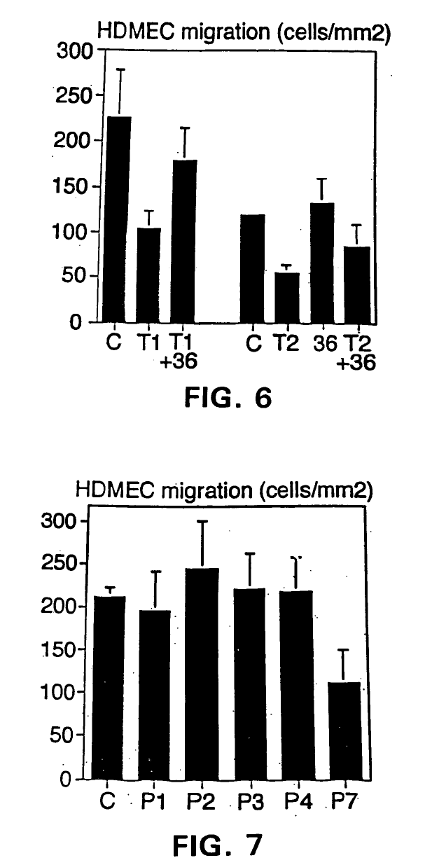

HDMEC migration experiments were performed essentially as described above. 1×10⁵ HDMEC were added to the upper chamber in 300 μl of DMEM medium, or in DMEM medium containing 10 μMol/l of the synthetic peptides. All media were supplemented with 10 mg/ml BSA. As shown in FIG. 7, in DMEM medium, 212±12 HDMEC/mm² migrated to the underside of the inserts (C; column 1). Peptides 1, 2, 3, and 4 did not significantly modify HDMEC migration. Peptide 2 (WSPWAEW) inhibited HDMEC migration by 47.6% (111±39 HDMEC/mm², column 2). These results reveal an important role of this TSP-2 specific peptide for the anti-angiogenic activity of TSP-2. Importantly, this peptide is distinct from the CSVTCG sequence that has been described to bind to the CD36 receptor on endothelial cells. Dawson et al. (1997) J. Cell. Biol. 138:707-717. All assays were performed in quadruplicate.

Biological Activities of TSP-2

Overexpression of TSP-2 in A431 cell xenotransplants potently decreased tumor growth, as compared to control tumors transfected with vector only. Tumor clones with the highest in vitro expression of TSP-2 demonstrated the most prominent growth inhibition in vivo. Similar results were obtained when TSP-2 overexpressing MeWo cells were transplanted into immunodeficient mice. Increased TSP-2 secretion by stable transfectants was confirmed by Western blot analyses of conditioned media. In situ hybridizations of tumor xenotransplants demonstrated that TSP-2 mRNA expression was maintained at high levels in TSP-2 transfected tumor cell clones. Together, these data provide evidence for a potent inhibitory effect of TSP-2 on skin cancer growth.

The tumor growth inhibition induced by TSP-2 in cutaneous squamous cell carcinomas was not due to direct TSP-2-mediated inhibition of tumor cell growth. Anchorage-independent cell growth, as determined by the ability to form colonies in soft agar, showed no significant differences between TSP-2 transfected A431 clones and control transfected A431 clones were detected. Results indicate that A431 cell growth is not influenced by TSP-2. Similarly, tumor treatment with the angiogenesis inhibitor angiostatin also led to reduced tumor size without changing tumor cell proliferation rates (O'Reilly et al. (1996) Nat Med 2:689-92).

Overexpression of TSP-2 in A431 xenotransplants resulted in extensive areas of tumor cell necrosis, possibly due to anti-angiogenic effects of TSP-2, to a reduced density of tumor blood vessels, and to reduced sizes of blood vessels. It is of interest that the first vascular changes observed during treatment of experimental tumors with an antibody to the angiogenesis factor VEGF consisted of a dramatic reduction of blood vessel diameters (Yuan et al. Proc Natl. Acad. Sci. USA 1996, 93:14765-70). Moreover, overexpression of VEGF in the skin of transgenic mice (Detmar et al. (1998) J. Invest Dennatol. 111:1-6) or in MEL-57 melanoma xenotransplants (Claffey et al. (1996) Cancer Res. 56:172-181) led to the development of tortuous and dilated blood vessels, and inhibition of the VEGF-inducible α1- and α2-integrins significantly inhibited VEGF-driven tumor angiogenesis in vivo, most prominently through reduction of average blood vessel diameters (Senger et al. (1997) Proc Natl. Acad. Sci. USA 94:13612-13617). To exclude that the reduction in vessel sizes observed in TSP-2 overexpressing A431 xenotransplant tumors was due to downregulation of VEGF expression, in situ hybridizations were performed of tumor xenotransplants. These studies demonstrated unchanged levels of VEGF mRNA expression in TSP-2 overexpressing tumors versus. controls. Therefore, the reduction in vessel sizes reflects an important biological activity of TSP-2 on the formation of tumor vasculature and demonstrates that similar vascular effects can be obtained by overexpression of TSP-2 or by inhibition of VEGF, suggesting antipodal roles of the two molecules in tumor angiogenesis.

In addition, it was found that significantly earlier development of skin papillomas and that increases numbers of skin papillomas and squamous cell carcinomas were found in TSP-2 deficient mice than in mice which express TSP-2.

In summary, TSP-2 induced a potent growth inhibition of malignant epithelial skin cancer. The anti-tumoral effect of TSP-2 was much more pronounced than the anti-tumoral effect of TSP-1, as compared in the identical A431 cell xenotransplant system. This effect was associated with significant inhibition of tumor angiogenesis.

Administration

TSP-2 can be administered to a subject by standard methods. For example, TSP-2 can be administered by any of a number of different routes including intravenous, intradermal, subcutaneous, oral (e.g., inhalation), transdermal (topical), transmucosal and rectal administration. In one embodiment, the TSP-2 agent can be administered topically. For example, the TSP-2 agent can be formulated such that it can be topically applied to an unwanted skin condition such as a skin neoplasm or psoriasis. In another embodiment, the TSP-2 agent can be administered orally. For example, the agent can be a retro-inverso peptide which is taken orally.

TSP, e.g., TSP-1 or TSP-2, can also be administered systemically or topically using a biocompatible controlled delivery system. For example, a TSP-2 protein, fragment, or analog can be administered to the subject in combination with a controlled release device, e.g., a biocompatible polymer, micro particle, or mesh. The device can reduce degradation and control the release of the TSP-2 protein, fragment, or analog. Such a TSP-2 biocompatible controlled release system can be administered to the subject, e.g., by injection or implantation, e.g., intramuscularly, subcutaneously, intravenously, or at an organ, joint cavity, or in situ at a lesion. Methods for controlled delivery of biologically active agents are known in the art and are described herein.

An agent which modulates TSP-2 activity, e.g., nucleic acid molecules, TSP-2 polypeptides, fragments or analogs, TSP-2 modulators, and anti-TSP-2 antibodies (also referred to herein as “active compounds”) can be incorporated into pharmaceutical compositions suitable for administration to a subject, e.g., a human. Such compositions typically include the nucleic acid molecule, polypeptide, modulator, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances are known. Except insofar as any conventional media or agent is incompatible with the active compound, such media can be used in the compositions of the invention. Supplementary active compounds can also be incorporated into the compositions.

A pharmaceutical composition can be formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELυ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a TSP-2 polypeptide or anti-TSP-2 antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Such transdermal formulations can by applied to the skin to treat inflammatory disorder of the skin such as psoriasis as well as skin neoplasias such as squamous cell carcinoma.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. One class of polymer systems for controlled release of polypeptides is based on polyethylene-co-vinyl acetate (EVA). Langer et al. ((1976) Nature 263:797-800) have shown that a wide variety of water-soluble macromolecules can be released for weeks and months from thin EVA matrices, formed by suspending macromolecular drug powder in an organic polymer solution and evaporating the solvent. Polymer blends displaying reverse phase morphology as described, e.g., in U.S. Pat. No. 4,795,641, can also be used. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Other methods of controlled delivery of an active agent, e.g., a TSP-2 protein, fragment, or analog, involve adding a second component or carrier to the active agent, typically in the form of a coating such that the coating acts to delay the release of the active agent in vivo (see, for example U.S. Pat. Nos. 4,060,598; 3,538,214; and 4,177,255). The active agent can also be dispersed in a gel formed from a monoglyceride and at least one vegetable oil, in amounts sufficient to form a reverse hexagonal liquid crystalline phase when in contact with an aqueous liquid (see, e.g., U.S. Pat. No. 5,143,934). Also useful are porous polymeric microparticles having preformed pores into which active agent is loaded (see, e.g., U.S. Pat. No. 5,470,582).

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals. Dosage forms to achieve sustained release are based on active agent diffusion through rate limiting barriers, chemical or enzymatic degradation of a drug carrier, combinations of diffusion and degradation, and mechanical or osmotic pumping of active agent (see, for example, U.S. Pat. No. 4,503,030).

The nucleic acid molecules described herein can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al., PNAS 91:3054-3057, 1994). The pharmaceutical preparation of the gene therapy vector can include the gene therapy vector in an acceptable diluent, or can include a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

The administration of an agent which increases a TSP-2 activity, e.g., a TSP-2 polypeptide can be repeated.

Combination Therapy

An agent which increases a TSP-2 activity can be administered alone or in combination with other agents. For example, in treating a subject having a disorder characterized by unwanted cell proliferation or angiogenesis, an agent which increases TSP-2 activity can be administered in combination with an agent which increases a TSP-1 activity. These agents can be administered simultaneously or sequentially. Generally any of the methods useful for increasing TSP-2 activity can be applied to TSP-1. For example, TSP-1 and TSP-2 activity can be increased by administering, e.g., a polypeptide, or a fragment or analog thereof; a nucleic acid that encodes a polypeptide, or a biologically active fragment or analog thereof; agonists, e.g., antibodies or small molecules; or combinations of the elements mentioned above.

Another agent which can be used in combination with a TSP-2 agent includes an agent which inhibits VEGF activity. VEGF activity can be decreased, e.g., by administering one or more of: a VEGF nucleic acid molecule, e.g., an antisense or VEGF ribozyme, that can bind to cellular VEGF nucleic acid sequence and inhibit expression of the protein; an antibody which specifically binds to VEGF protein; a dominant negative VEGF protein or fragment thereof; and an agent which decreases VEGF nucleic acid expression, e.g., a small molecule which binds the promoter of VEGF.

A chemotherapeutic agent can also be administered in combination with increasing a TSP-2 activity. Chemotherapeutic agents which can be administered include chosen from those disclosed below. Exemplary chemotherapeutic agents include: paclitaxel, vincristine, vinblastine, vindesine, vinorelbin, taxotere (Docetaxel), topotecan, camptothecin, irinotecan hydrochloride Camptosar, doxorubicin, etoposide, mitoxantrone, daunorubicin, idarubicin, teniposide, amsacrine, epirubicin, merbarone, piroxantrone hydrochloride, 5-fluorouracil, methotrexate, 6-mercaptopurine, 6-thioguanine, fludarabine phosphate, cytarabine (Ara-C), trimetrexate, gemcitabine, acivicin, alanosine, pyrazofurin, N-Phosphoracetyl-L-Asparate=PALA, pentostatin, 5-azacitidine, 5-Aza-2′-deoxycytidine, adenosine arabinoside (Ara-A), cladribine, ftorafur, UFI (combination of uracil and ftorafur), 5-fluoro-2′-deoxyuridine, 5-fluorouridine, 5′-deoxy-5-fluorouridine, hydroxyurea, dihydrolenchlorambucil, tiazofurin, cisplatin, carboplatin, oxaliplatin, mitomycin C, BCNU Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol, dihydrolenperone, spiromustine, geldenamycin, cytochalasins, depsipeptide, Lupron, ketoconazole, tamoxifen, goserelin (Zoledax), flutamide, 4′-cyano-3-(4-fluorophenylsulphonyl)-2-hydroxy-2-methyl-3′-(trifluoromethyl)propionanilide, Herceptin, anti-CD20 (Rituxan), interferon alpha, interferon beta, interferon gamma, interleukin 2, interleukin 4, interleukin 12, tumor necrosis factors, and radiation. Preferably, the chemotherapeutic agent is: paclitaxel (taxol), interferon alpha, gemcitabine, irinotecan, carboplatin, cisplatin, taxotere, doxorubicin, epirubicin, 5-fluorouracil, UFT, tamoxifen, goserelin, a HER2/neu antibody (e.g., Herceptin), anti-CD20, Lupron and flutamide.

Methods of increasing TSP-2 activity can be performed in conjunction with the administration of one or more of the above described agents. For example, TSP-1 and TSP-2 activity can be increased, or TSP-2 activity can be increased and VEGF activity decreased, or TSP-2 activity can be increased in conjunction with the administration of a chemotherapeutic agent. In one embodiment, TSP-2 activity can be increased in conjunction with the administration of two or more of the above described agents.

The administration of one or more of these agents can be repeated.

Analogs of TSP-2

nAnalogs can differ from naturally occurring TSP-2 in amino acid sequence or in ways that do not involve sequence, or both. Non-sequence modifications include in vivo or in vitro chemical derivatization of TSP-2. Non-sequence modifications include changes in acetylation, methylation, phosphorylation, carboxylation, or glycosylation.

Preferred analogs include TSP-2 (or biologically active fragments thereof) whose sequences differ from the wild-type sequence by one or more conservative amino acid substitutions or by one or more non-conservative amino acid substitutions, deletions, or insertions which do not abolish the TSP-2 biological activity. Conservative substitutions typically include the substitution of one amino acid for another with similar characteristics, e.g., substitutions within the following groups: valine, glycine; glycine, alanine; valine, isoleucine, leucine; aspartic acid, glutamic acid; asparagine, glutamine; serine, threonine; lysine, arginine; and phenylalanine, tyrosine. Other conservative substitutions can be taken from the table below.

TABLE 1
CONSERVATIVE AMINO ACID REPLACEMENTS

| For Amino | | |
|---|---|---|
| Acid | Code | Replace with any of |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, or |
| | | 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase peptide stability; such analogs may contain, for example, one or more non-peptide bonds (which replace the peptide bonds) in the peptide sequence. Also included are: analogs 5 that include residues other than naturally occurring L-amino acids, e.g., D-amino acids or non-naturally occurring or synthetic amino acids, e.g., β or γ amino acids; and cyclic analogs.

An analog in which one or more of the amino acids are D amino acids are also referred to herein as “retro-inverso polypeptides”. Retro-inverso polypeptides have been 10 used to increase the stability and/or biological activity of peptide sequences. See, e.g., Chover et al. (1993) Acc. Chem. Res. 26:266-273; Goodman et al. (1979) Acc. Chem. Res. 12:1-7. In one aspect, a TSP-2 polypeptide can be modified to include full or partial retro-inverso sequences. Such polypeptides can include polypeptide sequences described herein except that the sequence partially or entirely includes D-amino acids, thus having the reverse stoichemistry from a peptide synthesized using L amino acids. Retro-inverso analogs of TSP-2 can be prepared by conventional techniques described, for example, in Chover et al., supra, and Goodman et al., supra.

Retro-inverso polypeptides can decrease enzymatic degradation of a polypeptide. Thus, a retro-inverso polypeptide can be useful, for example, for oral administration because of the resistance of such polypeptides to enzymolysis.

TSP-2 analogs can be tested for their ability to inhibit unwanted proliferation, e.g., tumour growth, using the xenotransplant mouse model described herein.

Gene Therapy

The gene constructs of the invention can also be used as a part of a gene therapy protocol to deliver nucleic acids encoding either an agonistic or antagonistic form of a TSP-2 polypeptide. The invention features expression vectors for in vivo transfection and expression of a TSP-2 polypeptide in particular cell types so as to reconstitute the function of, or alternatively, antagonize the function of a TSP-2 polypeptide in a cell in which that polypeptide is misexpressed. Expression constructs of TSP-2 polypeptides, may be administered in any biologically effective carrier, e.g. any formulation or composition capable of effectively delivering the TSP-2 gene to cells in vivo. Approaches include insertion of the subject gene in viral vectors including recombinant retroviruses, adenovirus, adeno-associated virus, and herpes simplex virus-1, or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g. a cDNA, encoding a TSP-2 polypeptide. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, A. D. (1990) Blood 76:271). A replication defective retrovirus can be packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include ψCrip, ψCre, ψ2 and ψAm. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Another viral gene delivery system useful in the present invention utilizes adenovirus-derived vectors. The genome of an adenovirus can be manipulated such that it encodes and expresses a gene product of interest but is inactivated in terms of its ability to replicate in a normal lytic viral life cycle. See, for example, Berkner et al. (1988) BioTechniques 6:616; Rosenfeld et al. (1991) Science 252:431-434; and Rosenfeld et al. (1992) Cell 68:143-155. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they are not capable of infecting nondividing cells and can be used to infect a wide variety of cell types, including epithelial cells (Rosenfeld et al. (1992) cited supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al. cited supra; Haj-Ahmand and Graham (1986) J. Virol. 57:267).

Yet another viral vector system useful for delivery of the subject gene is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. (1992) Curr. Topics in Micro. and Immunol. 158:97-129). It is also one of the few viruses that may integrate its DNA into non-dividing cells, and exhibits a high frequency of stable integration (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). Vectors containing as little as 300 base pairs of AAV can be packaged and can integrate. Space for exogenous DNA is limited to about 4.5 kb. An AAV vector such as that described in Tratschin et al. (1985) Mol. Cell. Biol. 5:3251-3260 can be used to introduce DNA into cells. A variety of nucleic acids have been introduced into different cell types using AAV vectors (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790).

In addition to viral transfer methods, such as those illustrated above, non-viral methods can also be employed to cause expression of a TSP-2 polypeptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the subject TSP-2 gene by the targeted cell. Exemplary gene delivery systems of this type include liposomal derived systems, poly-lysine conjugates, and artificial viral envelopes. Other embodiments include plasmid injection systems, e.g., as described in Meuli et al. (2001) J Invest Dermatol. 116(1):131-135; Cohen et al. (2000) Gene Ther 7(22):1896-905; or Tam et al. (2000) Gene Ther 7(21): 1867-74.

In a representative embodiment, a gene encoding a TSP-2 polypeptide can be entrapped in liposomes bearing positive charges on their surface (e.g., lipofectins) and (optionally) which are tagged with antibodies against cell surface antigens of the target tissue (Mizuno et al. (1992) No Shinkei Geka 20:547-551; PCT publication W091/06309; Japanese patent application 1047381; and European patent publication EP-A-43075).

In clinical settings, the gene delivery systems for the therapeutic TSP-2 gene can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by Stereotactic injection (e.g. Chen et al. (1994) PNAS 91: 3054-3057).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced in tact from recombinant cells, e.g. retroviral vectors, the pharmaceutical preparation can comprise one or more cells which produce the gene delivery system.

Two Hybrid Systems

Two hybrid (interaction trap) assays can be used to identify a protein that interacts with TSP-2. These may include agonists, superagonists, and antagonists. (The subject protein and a protein it interacts with are used as the bait protein and fish proteins.). These assays rely on detecting the reconstitution of a functional transcriptional activator mediated by protein-protein interactions with a bait protein. In particular, these assays make use of chimeric genes which express hybrid proteins. The first hybrid comprises a DNA-binding domain fused to the bait protein. e.g., a TSP-2 molecule or a fragment thereof. The second hybrid protein contains a transcriptional activation domain fused to a “fish” protein, e.g. an expression library, e.g., an embryonic limb bud expression library. If the fish and bait proteins are able to interact, they bring into close proximity the DNA-binding and transcriptional activator domains. This proximity is sufficient to cause transcription of a reporter gene which is operably linked to a transcriptional regulatory site which is recognized by the DNA binding domain, and expression of the marker gene can be detected and used to score for the interaction of the bait protein with another protein.

Peptide Mimetics

The invention also provides for reduction of the protein binding domains of the subject TSP-2 polypeptides to generate mimetics, e.g. peptide or non-peptide agents. See, for example, “Peptide inhibitors of human papillomavirus protein binding to retinoblastoma gene protein” European patent applications EP-412,762A and EP-B31,080A.

Non-hydrolyzable peptide analogs of critical residues can be generated using benzodiazepine (e.g., see Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill, 1985), β-turn dipeptide cores (Nagai et al. (1985) Tetrahedron Lett 26:647; and Sato et al. (1986) J Chem Soc Perkin Trans 1:1231), and β-aminoalcohols (Gordon et al. (1985) Biochem Biophys Res Communl 26:419; and Dann et al. (1986) Biochem Biophys Res Commun 134:71).

Cell Therapy

TSP-2 can also be increased in a subject by introducing into a cell, e.g., a fibroblast or a keratinocyte, a nucleotide sequence that modulates the production of TSP-2, e.g., a nucleotide sequence encoding a TSP-2 polypeptide or functional fragment or analog thereof, a promoter sequence, e.g., a promoter sequence from a TSP-2 gene or from another gene; an enhancer sequence, e.g., 5′ untranslated region (UTR), e.g., a 5′ UTR from a TSP-2 gene or from another gene, a 3′ UTR, e.g., a 3′ UTR from a TSP-2 gene or from another gene; a polyadenylation site; an insulator sequence; or another sequence that modulates the expression of TSP-2. The cell can then be introduced into the subject.

Primary and secondary cells to be genetically engineered can be obtained from a variety of tissues and include cell types which can be maintained propagated in culture. For example, primary and secondary cells include fibroblasts, keratinocytes, epithelial cells (e.g., mammary epithelial cells, intestinal epithelial cells), endothelial cells, glial cells, neural cells, formed elements of the blood (e.g., lymphocytes, bone marrow cells), muscle cells (myoblasts) and precursors of these somatic cell types. Primary cells are preferably obtained from the individual to whom the genetically engineered primary or secondary cells are administered. However, primary cells may be obtained for a donor (other than the recipient) of the same species or another species (e.g., mouse, rat, rabbit, cat, dog, pig, cow, bird, sheep, goat, horse).

The term “primary cell” includes cells present in a suspension of cells isolated from a vertebrate tissue source (prior to their being plated i.e., attached to a tissue culture substrate such as a dish or flask), cells present in an explant derived from tissue, both of the previous types of cells plated for the first time, and cell suspensions derived from these plated cells. The term “secondary cell” or “cell strain” refers to cells at all subsequent steps in culturing. That is, the first time a plated primary cell is removed from the culture substrate and replated (passaged), it is referred to herein as a secondary cell, as are all cells in subsequent passages. Secondary cells are cell strains which consist of secondary cells which have been passaged one or more times. A cell strain consists of secondary cells that: 1) have been passaged one or more times; 2) exhibit a finite number of mean population doublings in culture; 3) exhibit the properties of contact-inhibited, anchorage dependent growth (anchorage-dependence does not apply to cells that are propagated in suspension culture); and 4) are not immortalized. A “clonal cell strain” is defined as a cell strain that is derived from a single founder cell. A “heterogenous cell strain” is defined as a cell strain that is derived from two or more founder cells.

Primary or secondary cells of vertebrate, particularly mammalian, origin can be transfected with an exogenous nucleic acid sequence which includes a nucleic acid sequence encoding a signal peptide, and/or a heterologous nucleic acid sequence, e.g., encoding TSP-2, and produce the encoded product stably and reproducibly in vitro and in vivo, over extended periods of time. A heterologous amino acid can also be a regulatory sequence, e.g., a promoter, which causes expression, e.g., inducible expression or upregulation, of an endogenous TSP-2 sequence. An exogenous nucleic acid sequence can be introduced into a primary or secondary cell by homologous recombination as described, for example, in U.S. Pat. No.: 5,641,670, the contents of which are incorporated herein by reference.

The transfected primary or secondary cells may also include DNA encoding a selectable marker which confers a selectable phenotype upon them, facilitating their identification and isolation. Methods for producing transfected primary and secondary cells which stably express exogenous synthetic DNA, clonal cell strains and heterogeneous cell strains of such transfected cells, methods of producing the clonal heterogeneous cell strains, and methods of treating or preventing an abnormal or undesirable condition through the use of populations of transfected primary or secondary cells are part of the present invention.

Transfection of Primary or Secondary Cells of Clonal or Heterogeneous Cell Strains

Vertebrate tissue can be obtained by standard methods such a punch biopsy or other surgical methods of obtaining a tissue source of the primary cell type of interest. For example, punch biopsy is used to obtain skin as a source of fibroblasts, keratinocytes, or endothelial cells. A mixture of primary cells is obtained from the tissue, using known methods, such as enzymatic digestion or explanting. If enzymatic digestion is used, enzymes such as collagenase, hyaluronidase, dispase, pronase, trypsin, elastase and chymotrypsin can be used.

The resulting primary cell mixture can be transfected directly or it can be cultured first, removed from the culture plate and resuspended before transfection is carried out. Primary cells or secondary cells are combined with exogenous nucleic acid sequence to, e.g., stably integrate into their genomes, and treated in order to accomplish transfection. The exogenous nucleic acid sequence can optionally include DNA encoding a selectable marker. The exogenous nucleic acid sequence and selectable marker-encoding DNA can either be on separate constructs or on a single construct. An appropriate quantity of DNA is used to ensure that at least one stably transfected cell containing and appropriately expressing exogenous DNA is produced. In general, approximately 0.1 to 500 μg of DNA is used.

As used herein, the term “transfection” includes a variety of techniques for introducing an exogenous nucleic acid into a cell including calcium phosphate or calcium chloride precipitation, microinjection, DEAE-dextrin-mediated transfection, lipofection or electrophoration.

Electroporation is carried out at approximate voltage and capacitance (and corresponding time constant) to result in entry of the DNA construct(s) into the primary or secondary cells. Electroporation can be carried out over a wide range of voltages (e.g., 50 to 2000 volts) and corresponding capacitance. Total DNA of approximately 0.1 to 500μg is generally used.

Methods such as calcium phosphate precipitation, modified calcium phosphate precipitation an polybrene precipitation, liposome fusion and receptor-mediated gene delivery can also be used to transect cells. Primary or secondary cells can also be transfected using microinjection. A stably, transfected cell can then be isolated and cultured and sub cultivated, under culturing conditions and for sufficient time to propagate stably transfected secondary cells an produce a clonal cell strain of transfected secondary cells. Alternatively, more than one transfected cell is cultured and sub cultured, resulting in production of a heterogeneous cell strain.

Transfected primary or secondary cells undergo sufficient number doubling to produce either a clonal cell strain or a heterogeneous cell strain of sufficient size to provide the therapeutic protein to an individual in effective amounts. In general, for example, O.0.1 cm² of skin is biopsies and assumed to contain 1,000,000 cells; one cell is used to produce a clonal cell strain and undergoes approximately 27 doublings to produce 100 million transfected secondary cells. If a heterogeneous cell strain is to be produced from an original transfected population of approximately 1000,000 cells, only 10 doublings are needed to produce 100 million transfected cells.

The number of required cells in a transfected clonal heterogeneous cell strain is variable and depends on a variety of factors, including but not limited to, the use of the transfected cells, the functional level of the exogenous DNA in the transfected cells, the site of implantation of the transfected cells (for example, the number of cells that can be used is limited by the anatomical site of implantation), and the age, surface area, and clinical condition of the patient. The put these factors in perspective, to deliver therapeutic levels of human growth hormone in an otherwise healthy 10 kg patient with isolated growth hormone deficiency, approximately one to five hundred million transfected fibroblast would be necessary (the volume of these cells is about that of the very tip of the patient's thumb).

Implantation of Clonal Cell Strain or Heterogeneous Cell Strain of Transfected Secondary Cells

The transfected cells, e.g., cells produced as described herein, can be introduced into an individual to whom the product is to be delivered. The clonal cell strain or heterogeneous cell strain is then introduced into an individual. Various routed of administration and various sites (e.g., renal sub capsular, subcutaneous, central nervous system (including intrathecal), intravascular, intrahepatic, intrasplanchnic, intraperitoneal (including intraomental), intramuscularly implantation) can be used. One implanted in individual, the transfected cells produce the product encoded by the heterologous DNA or are affected by the heterologous DNA itself. For example, an individual who suffers from a condition related to unwanted angiogenesis is a candidate for implantation of TSP-2 producing cells.

The individual can have a small skin biopsy performed; this is a simple procedure which can be performed on an outpatient basis. The piece of skin is taken, for example, from under the arm and can require about one minute to remove. The sample is processed, resulting in isolation of the patient's cell (e.g., fibroblasts) and genetically engineered to produce TSP-2 or another protein or molecule that induces the production of TSP-2. Based on the age, weight, and clinical condition of the patient, the required number of cells are grown in large-scale culture. The entire process should require 4-6 weeks and, at the end of that time, the appropriate number of genetically engineered cells are introduced into the individual, once again as an outpatient (e.g., by injecting them back under the patient's skin, e.g., on the scalp or face). The patient is now capable of producing TSP-2 which can ameliorate symptoms of hair loss.

For some, this will be a one-time treatment and, for others, multiple cell therapy treatments will be required.

As this example suggests, the cells used will generally be patient-specific genetically engineered cells. It is possible, however, to obtain cells from another individual of the same species or from a different species. Use of such cells might require administration of an immunosuppressant, alteration of histocompatibility antigens, or use of a barrier device to prevent rejection of the implanted cells.

Transfected primary or secondary cells can be administered alone or in conjunction with a barrier or agent for inhibiting immune response against the cell in a recipient subject. For example, an immunosuppressive agent can be administered to a subject to inhibit or interfere with normal response in the subject. Preferably, the immunosuppressive agent is an immunosuppressive drug which inhibits T cell/or B cell activity in a subject. Examples of such immunosuppressive drugs commercially available (e.g., cyclosporin A is commercially available from Sandoz Corp. East Hanover, N.J.).

An immunosuppressive agent, e.g., drug, can be administered to a subject at a dosage sufficient to achieve the desired therapeutic effect (e.g., inhibition of rejection of the cells). Dosage ranges for immunosuppressive drugs are known in the art. See, e.g., Freed et al. (1992) N. Engl. J. Med. 327:1549; Spencer et al. (1992) N. Engl. J. Med. 327:1541' Widner et al. (1992) n. Engl. J. Med. 327:1556). Dosage values may vary according to factors such as the disease state, age, sex, and weight of the individual.

Another agent with can be used to inhibit T cell activity in a subject is an antibody, or fragment of derivative thereof. Antibodies capable of depleting or sequestering T cells in vivo are known in the art. Polyclonal antisera can be used, for example, anti-lymphocyte serum. Alternatively, one or more monoclonal antibodies can be used. Preferred T cell depleting antibodies include monoclonal antibodies which bind to CD2, CD3, CD4, CD8, CD40, CD40, ligand on the cell surface. Such antibodies are known in the art and are commercially available, for example, from American Type Culture Collection. A preferred antibody for binding CD3 on human T cells is OKT3 (ATCC CRL 8001).

An antibody which depletes, sequesters or inhibits T cells within a recipient subject can be administered in a dose for an appropriate time to inhibit rejection of cells upon transplantation. Antibodies are preferably administered intravenously in a pharmaceutically acceptable carrier of diluent (e.g., saline solution).

An advantage of the use of transfected or secondary cells is that by controlling the number of cells introduced into an individual, one can control the amount of the protein delivered to the body. In addition, in some cases, it is possible to remove the transfected cells of there is no longer a need for the product. A further advantage of treatment by use of transfected primary or secondary cells of the present invention is that production of the therapeutic product can be regulated, such as through the an administration of zinc, steroids or an agent which affects transcription of a protein, product or nucleic acid product or affects the stability of a nucleic acid product.

Antibodies

The invention also includes antibodies specifically reactive with a subject TSP-2 polypeptides. Anti-protein/anti-peptide antisera or monoclonal antibodies can be made as described herein by using standard protocols (See, for example, Antibodies: A Laboratory Manual ed. by Harlow and Lane (Cold Spring Harbor Press: 1988)).

Antibodies which specifically bind TSP-2 epitopes can also be used in immunohistochemical staining of tissue samples in order to evaluate the abundance and pattern of expression of TSP-2. Anti-TSP-2 antibodies can be used diagnostically in immuno-precipitation and immuno-blotting to detect and evaluate TSP-2 levels in tissue or bodily fluid as part of a clinical testing procedure.

Another application of antibodies of the present invention is in the immunological screening of cDNA libraries constructed in expression vectors such as λgt 11, λgt18-23, λZAP, and λORF8. Messenger libraries of this type, having coding sequences inserted in the correct reading frame and orientation, can produce fusion proteins. For instance, λgt11 will produce fusion proteins whose amino termini consist of β-galactosidase amino acid sequences and whose carboxy termini consist of a foreign polypeptide. Antigenic epitopes of a subject polypeptide can then be detected with antibodies, as, for example, reacting nitrocellulose filters lifted from infected plates with antibodies of the invention. Phage, scored by this assay, can then be isolated from the infected plate. Thus, the presence of homologs can be detected and cloned from other animals, and alternate isoforms (including splicing variants) can be detected and cloned from human sources.

### Other Embodiments

It is understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All patents and references cited herein are incorporated in their entirety by reference.

1-55. (canceled)

56. A method of treating a subject having an angiogenesis-dependent tumor, the method comprising:
identifying a subject having an angiogenesis-dependent tumor; and
administering to the subject a polypeptide comprising a fragment of SEQ ID NO:2 (TSP-2) or a sequence at least 95% identical to SEQ ID NO:2, wherein the fragment is capable of inhibiting endothelial cell migration, and comprises at least 10 amino acids of SEQ ID NO:2 or the sequence at least 95% identical to SEQ ID NO:2.

57. The method of claim 56, wherein the polypeptide comprises at least 10 contiguous amino acids of (a) a procollagen domain of TSP-2, or (b) a type I repeat of TSP-2.

58. The method of claim 56, wherein the fragment comprises the sequence of SEQ ID NO:10 (WSPWAEW).

59. The method of claim 56, wherein the tumor is an epithelial tissue tumor, a skin tumor, a prostate tumor, a colon tumor, a breast tumor, a lung tumor, or a Kaposi's sarcoma.

60. The method of claim 56, further comprising increasing TSP-1 activity, inhibiting VEGF activity, or administering a chemotherapeutic agent.

61. The method of claim 56, wherein the fragment is at least 50 amino acids in length.

62. The method of claim 57, wherein the fragment comprises one type I repeat.

63. The method of claim 57, wherein the fragment comprises between about 5 to 50 amino acids of a type I repeat.

64. The method of claim 56, wherein the fragment comprises at least one sequence selected from the group of: amino acids 382-429 of SEQ ID NO:2, amino acids 438-490 of SEQ ID NO:2, and amino acids 495-547 of SEQ ID NO:2.

65. The method of claim 56, wherein the fragment comprises SEQ ID NO:11 (CSVTVG).

66. The method of claim 57, wherein the fragment comprises a procollagen domain or a fragment thereof.

67. The method of claim 56, wherein the fragment comprises two type I repeats.

68. The method of claim 56, wherein the fragment comprises three type I repeats.

69. The method of claim 56, wherein the fragment comprises an amino acid sequence encoded by nucleotides 294-1367 of SEQ ID NO:1.

70. The method of claim 56, wherein the fragment comprises an amino acid sequence encoded by nucleotides 294-1883 of SEQ ID NO:1.

71. The method of claim 56, wherein the fragment comprises an amino acid sequence encoded by nucleotides 1383-1883 of SEQ ID NO:1.

72. The method of claim 56, wherein the polypeptide comprises TSP-2 (SEQ ID NO:2).

73. The method of claim 72, wherein the polypeptide consists of TSP-2 (SEQ ID NO:2).

74. A method of treating an angiogenesis-dependent tumor, the method comprising administering to the subject a fragment of TSP-2 comprising the sequence of SEQ ID NO: 10 (WSPWAEW).

75. A method of treating a subject having an angiogenesis-dependent tumor, the method comprising:
identifying a subject having an angiogenesis-dependent tumor; and
administering to the subject a cell genetically engineered to express TSP-2 (SEQ ID NO:2) or a fragment thereof, capable of inhibiting endothelial cell migration, wherein the fragment comprises at least 10 amino acids of TSP-2.

76. The method of claim 75, wherein the TSP-2 or fragment thereof comprises at least 10 contiguous amino acids of (a) a procollagen domain of TSP-2, or (b) a type I repeat of TSP-2.

77. The method of claim 75, wherein the cell is selected from the group consisting of: a fibroblast, a keratinocyte, an endothelial cell, a glial cell, a neural cell, a lymphocyte, a bone marrow cell, and a muscle cell.

78. The method of claim 75, wherein the tumor is selected from the group consisting of: melanoma, a prostate tumor, a breast tumor, a colon tumor, and a lung tumor.

79. The method of claim 75, wherein the subject is a human.

80. The method of claim 76, wherein the cell is genetically engineered to express a fragment of TSP-2 capable of inhibiting endothelial cell migration, wherein the fragment comprises at least 10 contiguous amino acids of a type I repeat of TSP-2.
